(19) United States
(12) Patent Application Publication
(10) Pub. No.: US 2009/0225585 A1
(43) Pub. Date: Sep. 10, 2009

(54) Self-Contained Charge Storage Molecules for Use in Molecular Capacitors

(76) Inventor: J. Adrian Hawkins, Denver, CO (US)

Correspondence Address: MORGAN, LEWIS & BOCKIUS, LLP. (PA), 2 PALO ALTO SQUARE, 3000 EL CAMINO REAL, SUITE 700, PALO ALTO, CA, 94306, US

(21) Appl. No.: 12/344,090

(22) Filed: Dec. 24, 2008

Related U.S. Application Data
(60) Provisional application No. 61/017,084, filed on Dec. 27, 2007.

(51) Int. Cl. G11C 13/00; C07D 487/22; H01G 9/025

(52) U.S. Cl. 365/151; 540/145; 361/525

(57) ABSTRACT

The invention encompasses self-contained charge storage molecules for use in memory devices such as for example static, permanent and dynamic random access memory. In particular, the invention encompasses molecules possessing structural features, which allow such molecules to form self-contained charge storage units for use, for example, in a molecular capacitor. The invention further encompasses operational systems comprising such self-contained charge storage molecules.

### 1. CROSS REFERENCE TO RELATED APPLICATIONS

This patent application claims the benefit of, and priority to, U.S. Provisional Patent Application Ser. No. 61/017,084, filed on Dec. 27, 2007, entitled “Self-Contained Charge Storage Molecules for Use in Molecular Capacitors” the entire disclosure of which is hereby incorporated by reference.

### 2. FIELD OF THE INVENTION

The invention encompasses self-contained charge storage molecules for use in memory devices such as for example static, permanent and dynamic random access memory. In particular, the invention encompasses molecules possessing structural features, which allow such molecules to form self-contained charge storage units for use, for example, in a molecular capacitor. The invention further encompasses operational systems comprising such self-contained charge storage molecules.

### 2. BACKGROUND OF THE INVENTION

The continued miniaturization of information processing systems requires the development of components with features of sizes less than 0.1 μm. While such feature sizes will most likely be achieved with projected technologies, it is uncertain whether devices that rely on the bulk properties of semiconductor materials will retain the required functional characteristics at these dimensions. An illustrative example is the design of information storage systems such as dynamic random access memory (DRAM). The basic DRAM cell consists of one capacitor and one transistor, where the charge stored on the capacitor indicates the bit level (i.e., either 0 or 1).

In many cases improvements in semiconductor processing technology have had the effect of improving important figures to make denser, larger, faster and more power efficient memory devices. In many cases, the solid state electronic behavior of the devices improves as the devices become smaller. Unfortunately, conventional silicon-based memory, such as DRAM memory, has reached a point where continued reduction in the size of conventional semiconductor memory cells is expected to adversely affect at least some of these important parameters.

One reason for the reduced speed and increased power consumption at smaller dimensions is that memory devices usually implement a capacitor for each stored bit of information. A capacitor is a charge storage device formed by conductive plates that are separated by an insulator. As capacitors become smaller the quantity of charge that can be stored is reduced. To serve as a reliable memory device, a capacitor must have sufficient capacity to hold a signal at a level that can be later reliable detected as data. Moreover, capacitors are inherently “leaky” devices in that some of the charge stored in a capacitor dissipates or leaks over time. Memory devices based on smaller capacitors are more sensitive to leakage problems because they simply have less charge that can be lost before the stored data becomes irretrievable.

To overcome the transient nature of capacitive storage, memory devices use refresh circuitry that frequently reads out a stored signal, amplifies it to a higher level, and stores it back into the capacitor. As a capacitor shrinks, the rate at which the capacitor needs to be refreshed increases. In turn, higher capacitor refresh rates reduce the percentage of time that a memory cell is available for reading and writing data. Moreover, a greater percentage of the total power consumption of the memory device is then used to refresh the memory. Even when the device is in a dormant or inactive state, traditional DRAM requires continuous refreshing and therefore continuous power consumption. Accordingly, researchers are actively seeking new ways of storing data signals that overcome the problems associated with smaller capacitors in conventional capacitor based memory devices.

Memory cell designers have attempted to maintain low refresh rates in smaller memory cells by boosting the amount of capacitance that can be formed in a given amount of chip area. Boosting capacitance often involves increasing the surface area of the capacitor's charge holding material, which is very difficult to do when the overall size of the capacitor is shrinking. While designers have had some success at controlling surface area by forming the charge holding material into three-dimensional trench and stacked capacitor designs, it is unlikely that these techniques can be relied for continued progress rendering larger capacitances in smaller devices. The solid state electronic behaviors upon which device performance is predicated begin to break down as the dimensions of various device features become smaller such that a capacitor can no longer store sufficient charge for sufficient time to be useful in a memory device.

Another problem facing memory designers trying to increase information density (e.g., the amount of information that can be stored in a given area of the memory chip). Each memory cell of a conventional solid state capacitor can only store one bit of information. Accordingly, it would be desirable to have a memory device with improved information storage density achieved by having a memory cell that can reliably store a plurality of discrete states.

Molecular memory is based on the properties of specially-designed molecules that store information by adding or removing electrons and then detecting the charge state of the molecule. The molecules can be oxidized and reduced (i.e., electrons removed or replaced) in a way that is stable, reproducible, and reversible. In this way, molecules can be used as reliable memory locations for electronic devices. In many ways, each molecule acts like an individual capacitor device, similar to a conventional capacitor, but storing only a few electrons of charge, which are accessible only at specific, quantized voltage levels. A key difference between molecular memory and conventional memory is that as the size of a memory element becomes smaller, the properties of semiconductor or polymer materials change in undesirable ways, while the properties of our molecular capacitors remain the same. This allows scaling to very small size elements.

To exploit molecular memory, molecules preferably have properties needed for a particular application. The most important property is the oxidation potentials (i.e., the energy required to remove one or more electrons). This energy is a quantum mechanical property of the whole molecule and is typically between 100 and 200 mV for each electron removed. The value is exact and doesn't vary.

Another property that can be designed into molecules is chemical self-assembly. This allows the molecules to attach only to a particular type of surface (for example, gold, silicon, various metals and oxides), to pack tightly on that surface, and to align properly on the surface for electronic operation. By experimenting with chemical self-assembly, molecular memory chips can be manufactured using equipment and processes common in the semiconductor industry. Molecules could be applied to an entire wafer by spraying or dipping and attach only to those exposed surfaces they are designed for. Unattached molecules are simply washed away from the other surfaces.

In view of the above, it is apparent that a need exists for novel compositions for molecular memory devices that overcome limitations imposed by conventional solid state memory design. In particular, there is a need for molecular memory cells, molecular memory arrays, and electronic devices including molecular memory.

### 3. SUMMARY OF THE INVENTION

The invention broadly encompasses a general design for novel self-contained charge storage molecules for use in molecular capacitors, illustrative molecules possessing such features, the use of molecules in operational systems, and descriptions of such operational systems.

In one embodiment, the invention encompasses a class of molecules that incorporate an oxidizable component, a reducible component, and an ionic conducting electrolyte.

In another embodiment, the invention encompasses one or more self-contained charge storage compositions comprising one or more molecules comprising an oxidizable component, a reducible component and an ionic conducting electrolyte positioned between a working electrode and a counter electrode capable of affording electrical capacitance.

In another embodiment the invention encompasses a molecular memory element that includes a switching device, a bit line and a word line coupled to the switching device and a molecular storage device accessible through the switching device. The molecular storage device is capable of being placed in two or more discrete states, wherein the molecular storage device is placed in one of the discrete states by signals applied to the bit and word line. The molecular storage device comprises a first electrode, a second electrode and a molecular material between the first and second electrode.

Another embodiment encompasses molecular memory arrays comprising a plurality of molecular storage elements where each molecular storage element is capable of being placed in two or more discrete states. A plurality of bit lines and word lines are coupled to the plurality of molecular storage elements such that each molecular storage element is coupled to and addressable by at least one bit line and at least one word line.

In another embodiment, the invention encompasses a molecular memory device comprising an addressable array of molecular storage elements. An address decoder receives a coded address and generates word line signals corresponding to the coded address. A word line driver is coupled to the address decoder and produces amplified word line signals. The amplified word line signals control switches that selectively couple members of the array of molecular storage elements to bit lines. Read/write logic coupled to the bit lines determines whether the molecular memory device is in a read mode or a write mode. In a read mode, sense amplifiers coupled to each bit line detect an electronic state of the selectively coupled molecular storage elements and produce a data signal on the bit line indicative of the electronic state of the selectively coupled molecular storage elements. In a write mode, the read/write logic drives a data signal onto the bit lines and the selectively coupled molecular storage elements.

Another embodiment encompasses devices including logic integrated with embedded molecular memory devices such as application specific integrated circuit (ASIC) and system on chip (SOC) devices and the like. Such implementations comprise one or more functional components formed monolithically with and interconnected to molecular memory devices. The functional components may comprise solid state electronic devices and/or molecular electronic devices.

In particular embodiments, the molecular storage device is implemented as a stacked structured formed subsequent to and above a semiconductor substrate having active devices formed therein. In other embodiments, the molecular storage device is implemented as a micron or nanometer sized hole in a semiconductor substrate have active devices formed therein. The molecular storage device is fabricated using processing techniques that are compatible with the semiconductor substrate and previously formed active devices in the semiconductor substrate. The molecular storage device comprises, for example, an electrochemical cell having two or more electrode surfaces separated by an electrolyte (e.g., a ceramic or solid electrolyte). Storage molecules (e.g., molecules having one or more oxidation states that can be used for storing information) are coupled to an electrode surface within the electrochemical cells.

Other embodiments of the invention include the use of components independently selected from transistor switching devices including field effect transistor; a row decoder coupled to the word line; a column decoder coupled to the bit line; a current preamplifier connected to the bit line; a sense amplifier connected to the bit line, an address decoder that receives a coded address and generates word line signals corresponding to the coded address, a line driver coupled to the address decoder wherein the line driver produces amplified word line signals (optionally wherein the amplified word line signals control switches that selectively couple members of the array of molecular storage elements to bit lines), read/write logic coupled to the bit lines, wherein the read/write logic determines whether the molecular memory devices is in a read mode or a write mode, sense amplifiers coupled to each bit line, wherein when the device is in a read mode, sense amplifiers coupled to each bit line detect an electronic state of the selectively coupled molecular storage elements and produce a data signal on the bit line indicative of the electronic state of the selectively coupled molecular storage elements (such that when the device is in a write mode, the read/write logic drives a data signal onto the bit lines and the selectively coupled molecular storage elements) electrolyte layers; and combinations thereof.

Further embodiments encompass the second electrode being coupled to ground, and the bit and word lines being either perpendicular or parallel.

Additional embodiments have the memory arrays of the invention comprising volatile memory such as DRAM or SRAM, or non-volatile memory such as Flash or ferroelectric memory.

A further embodiment provides arrays wherein the molecular storage device comprises an attachment layer formed on the first electrode, wherein the attachment layer comprises an opening and wherein the molecular material is in the opening and electronically coupled to the second electrode layer and an electrolyte layer formed on the attachment layer.

Another embodiment encompasses a monolithically integrated device comprising logic devices configured to perform a particular function and embedded molecular memory devices of the invention coupled to the logic devices. The device may optionally comprise an application specific integrated circuit (ASIC), a system on chip (SOC), a solid state electronic devices or molecular electronic devices.

### 4. BRIEF DESCRIPTION OF THE DRAWINGS

FIG. 1 illustrates the operation/functionality of a self-contained charge storage unit in accordance with some embodiments of the present invention;

FIG. 2 illustrates molecular memory in a liquid electrolyte system in accordance with some embodiments of the present invention;

FIG. 3 illustrates molecular memory in a solid system in accordance with other embodiments of the present invention;

FIGS. 4A, 4B and 4C depict schematic views of various embodiments of the present invention;

FIG. 5 depicts a specific phosphine example of the present invention; and

FIG. 6 depicts a generic phosphine example in accordance with the present invention.

### 5. DETAILED DESCRIPTION OF INVENTION

### 5.1. Definitions

As used herein and unless otherwise indicated, addressing a particular element refers to associating (e.g., electrically coupling) that memory element with an electrode such that the electrode can be used to specifically determine the oxidation state(s) of that memory element.

As used herein and unless otherwise indicated, the term “acyl” refers to an organic acid group in which the OH of the carboxyl group is replaced by some other substituent (RCO—), such as described herein as “R” substitutent groups. Examples include, but are not limited to, halo, acetyl and benzoyl.

As used herein and unless otherwise indicated, the term “alkoxy group” means an —O-alkyl group, wherein alkyl is as defined herein. An alkoxy group can be unsubstituted or substituted with one or two suitable substituents. Preferably, the alkyl chain of an alkyloxy group is from 1 to 6 carbon atoms in length, referred to herein, for example, as “(C1-C6)alkoxy.”

As used herein and unless otherwise indicated, “alkyl” by itself or as part of another substituent, refers to a saturated or unsaturated, branched, straight-chain or cyclic monovalent hydrocarbon radical derived by the removal of one hydrogen atom from a single carbon atom of a parent alkane, alkene or alkyne. Also included within the definition of an alkyl group are cycloalkyl groups such as C5 and C6 rings, and heterocyclic rings with nitrogen, oxygen, sulfur or phosphorus (heterocycloalkyl). Alkyl also includes heteroalkyl, with heteroatoms of sulfur, oxygen, nitrogen, and silicon finding particular use in certain embodiments. Alkyl groups can be optionally substituted with R groups, independently selected at each position as described below.

Examples of alkyl groups include, but are not limited to, (C1-C6)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl.

The term “alkyl” is specifically intended to include groups having any degree or level of saturation, i.e., groups having exclusively carbon-carbon single bonds, groups having one or more carbon-carbon double bonds, groups having one or more carbon-carbon triple bonds and groups having mixtures of single, double and triple carbon-carbon bonds. Where a specific level of saturation is intended, the expressions “alkanyl,” “alkenyl,” and “alkynyl” are used. Preferably, an alkyl group comprises from 1 to 20 carbon atoms, more preferably, from 1 to 10 carbon atoms, most preferably, from 1 to 6 carbon atoms.

“Alkanyl” by itself or as part of another substituent, refers to a saturated branched, straight-chain or cyclic alkyl radical derived by the removal of one hydrogen atom from a single carbon atom of a parent alkane. “Heteroalkanyl” is included as described above.

“Alkenyl” by itself or as part of another substituent, refers to an unsaturated branched, straight-chain or cyclic alkyl radical having at least one carbon-carbon double bond derived by the removal of one hydrogen atom from a single carbon atom of a parent alkene. The group may be in either the cis or trans conformation about the double bond(s). Suitable alkenyl groups include, but are not limited to (C2-C6)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or more independently selected R groups.

“Alkynyl” by itself or as part of another substituent, refers to an unsaturated branched, straight-chain or cyclic alkyl radical having at least one carbon-carbon triple bond derived by the removal of one hydrogen atom from a single carbon atom of a parent alkyne.

Also included within the definition of “alkyl” is “substituted alkyl”. “Substituted” is usually designated herein as “R”, and refers to a group in which one or more hydrogen atoms are independently replaced with the same or different substituent(s). R substituents can be independently selected from, but are not limited to, hydrogen, halogen, alkyl (including substituted alkyl (alkylthio, alkylamino, alkoxy, etc.), cycloalkyl, substituted cycloalkyl, cycloheteroalkyl, and substituted cycloheteroalkyl), aryl (including substituted aryl, heteroaryl or substituted heteroaryl), carbonyl, alcohol, amino, amido, nitro, ethers, esters, aldehydes, sulfonyl, sulfoxyl, carbamoyl, acyl, cyano, thiocyanato, silicon moieties, halogens, sulfur containing moieties, phosphorus containing moieties, etc. In some embodiments, as described herein, R substituents include redox active moieties (ReAMs). In some embodiments, optionally R and R′ together with the atoms to which they are bonded form a cycloalkyl (including cycloheteroalkyl) and/or cycloaryl (including cycloheteroaryl), which can also be further substituted as desired. In the structures depicted herein, R is hydrogen when the position is unsubstituted. It should be noted that some positions may allow two or three substitution groups, R and R′, in which case the R and R′ groups may be either the same or different.

In some embodiments, the R groups (subunits) are used to adjust the redox potential(s) of the subject compound. Thus, as is more fully described below and in references cited herein, an R group such as a redox active subunit can be added to a macrocycle, particularly a porphyrinic macrocycle to alter its redox potential. Certain preferred substituents include, but are not limited to, 4-chlorophenyl, 3-acetamidophenyl, 2,4-dichloro-4-trifluoromethyl, and ferrocene (including ferrocene derivatives). When the substituents are used for altering redox potentials, preferred substituents provide a redox potential range of less than about 5 volts, preferably less than about 2 volts, more preferably less than about 1 volt.

In certain embodiments, the R groups are as defined and depicted in the figures and the text from U.S. 60/687,464 which is incorporated herein by reference. A number of suitable proligands and complexes, as well as suitable substituents, are outlined in U.S. Pat. Nos. 6,212,093; 6,728,129; 6,451,942; 6,777,516; 6,381,169; 6,208,553; 6,657,884; 6,272,038; 6,484,394; and U.S. Ser. Nos. 10/040,059; 10/682,868; 10/445,977; 10/834,630; 10/135,220; 10/723,315; 10/456,321; 10/376,865; all of which are expressly incorporated by reference, in particular for the structures and descriptions thereof depicted therein, hereby expressly incorporated as substituent embodiments, both for the particular macrocycle the substituents are depicted within and for further substituted derivatives.

By “aryl” or grammatical equivalents herein is meant an aromatic monocyclic or polycyclic hydrocarbon moiety generally containing 5 to 14 carbon atoms (although larger polycyclic rings structures may be made) and any carbocyclic ketone or thioketone derivative thereof, wherein the carbon atom with the free valence is a member of an aromatic ring. Aromatic groups include arylene groups and aromatic groups with more than two atoms removed. For the purposes of this application aryl includes heteroaryl. “Heteroaryl” means an aromatic group wherein 1 to 5 of the indicated carbon atoms are replaced by a heteroatom chosen from nitrogen, oxygen, sulfur, phosphorus, boron and silicon wherein the atom with the free valence is a member of an aromatic ring, and any heterocyclic ketone and thioketone derivative thereof. Thus, heterocycle includes both single ring and multiple ring systems, e.g. thienyl, furyl, pyrrolyl, pyrimidinyl, indolyl, purinyl, quinolyl, isoquinolyl, thiazolyl, imidazolyl, naphthalene, phenanthroline, etc. Also included within the definition of aryl is substituted aryl, with one or more substitution groups “R” as defined herein and outlined above and herein. For example, “perfluoroaryl” is included and refers to an aryl group where every hydrogen atom is replaced with a fluorine atom. Also included is oxalyl.

The term “halogen” refers to one of the electronegative elements of group VIIA of the periodic table (fluorine, chlorine, bromine, iodine, astatine).

The term “nitro” refers to the NO.sub.2 group.

By “amino groups” or grammatical equivalents herein is meant —NH2, —NHR and —NRR′ groups, with R and R′ independently being as defined herein.

The term “pyridyl” refers to an aryl group where one CH unit is replaced with a nitrogen atom.

The term “cyano” refers to the —CN group.

The term “thiocyanato” refers to the —SCN group.

The term “sulfoxyl” refers to a group of composition RS(O)— where R is some substitution group as defined herein, including alkyl, (cycloalkyl, perfluoroalkyl, etc.), or aryl (e.g., perfluoroaryl group). Examples include, but are not limited to methylsulfoxyl, phenylsulfoxyl, etc.

The term “sulfonyl” refers to a group of composition RSO2- where R is a substituent group, as defined herein, with alkyl, aryl, (including cycloalkyl, perfluoroalkyl, or perfluoroaryl groups). Examples include, but are not limited to methylsulfonyl, phenylsulfonyl, p-toluenesulfonyl, etc.

The term “carbamoyl” refers to the group of composition R(R′)NC(O)— where R and R′ are as defined herein, examples include, but are not limited to N-ethylcarbamoyl, N,N-dimethylcarbamoyl, etc.

The term “amido” refers to the group of composition R.sup.1CON(R.sup.2)- where R.sup.1 and R.sup.2 are substituents as defined herein. Examples include, but are not limited to acetamido, N-ethylbenzamido, etc.

The term “acyl” refers to an organic acid group in which the OH of the carboxyl group is replaced by some other substituent (RCO—). Examples include, but are not limited to acetyl, benzoyl, etc.

In certain embodiments, when a metal is designated, e.g., by “M” or “Mn”, where n is an integer, it is recognized that the metal can be associated with a counterion.

As used herein and unless otherwise indicated, the term “amperometric device” is a device capable of measuring the current produced in an electrochemical cell as a result of the application of a specific field potential (“voltage”).

As used herein and unless otherwise indicated, the term “aryloxy group” means an —O-aryl group, wherein aryl is as defined herein. An aryloxy group can be unsubstituted or substituted with one or two suitable substituents. Preferably, the aryl ring of an aryloxy group is a monocyclic ring, wherein the ring comprises 6 carbon atoms, referred to herein as “(C6)aryloxy.”

As used herein and unless otherwise indicated, the term “benzyl” means —CH2-phenyl.

As used herein and unless otherwise indicated, the term “carbonyl” group is a divalent group of the formula —C(O)—.

As used herein and unless otherwise indicated, the term “coulometric device” is a device capable of measuring the net charge produced during the application of a potential field (“voltage”) to an electrochemical cell.

As used herein and unless otherwise indicated, the term “cyano” refers to the —CN group.

As used herein and unless otherwise indicated, the term “different and distinguishable” when referring to two or more oxidation states means that the net charge on the entity (atom, molecule, aggregate, subunit, etc.) can exist in two different states. The states are said to be “distinguishable” when the difference between the states is greater than thermal energy at room temperature (e.g., 0° C. to about 40° C.).

As used herein and unless otherwise indicated, the term “double-decker sandwich coordination compound” refers to a sandwich coordination compound as described herein where n is 2, thus having the formula L′-M′-LZ, wherein each of L1 and LZ may be the same or different (see, e.g., Jiang et al. (1999) J. Porphyrins Phthalocyanines 3: 322-328) and U.S. Pat. Nos. 6,212,093; 6,451,942; 6,777,516; and polymerization of these molecules is described in U.S. Publication No. 20070123618, hereby incorporated by reference in its entirety.

As used herein and unless otherwise indicated, the term “E_{1/2}” refers to the practical definition of the formal potential (Bₒ) of a redox process as defined by B−Bₒ+(RT/nF)ln(Dₒₓ/D_{red}) where R is the gas constant, T is temperature in K (Kelvin), n is the number of electrons involved in the process, F is the Faraday constant (96,485 Coulomb/mole), Dₒₓ is the diffusion coefficient of the oxidized species and D_{red} is the diffusion coefficient of the reduced species.

As used herein and unless otherwise indicated, the term “electrically coupled” when used with reference to a storage molecule and/or storage medium and electrode refers to an association between that storage medium or molecule and the electrode such that electrons move from the storage medium/molecule to the electrode or from the electrode to the storage medium/molecule and thereby alter the oxidation state of the storage medium/molecule. Electrical coupling can include direct covalent linkage between the storage medium/molecule and the electrode, indirect covalent coupling (e.g. via a linker), direct or indirect ionic bonding between the storage medium/molecule and the electrode, or other bonding (e.g. hydrophobic bonding). In addition, no actual bonding may be required and the storage medium/molecule may simply be contacted with the electrode surface. There also need not necessarily be any contact between the electrode and the storage medium/molecule where the electrode is sufficiently close to the storage medium/molecule to permit electron tunneling between the medium/molecule and the electrode.

As used herein and unless otherwise indicated, the term “electrochemical cell” consists minimally of a reference electrode, a working electrode, a redox-active medium (e.g. a storage medium), and, if necessary, some means (e.g., a dielectric) for providing electrical conductivity between the electrodes and/or between the electrodes and the medium. In some embodiments, the dielectric is a component of the storage medium.

As used herein and unless otherwise indicated, the term “electrode” refers to any medium capable of transporting charge (e.g., electrons) to and/or from a storage molecule. Preferred electrodes are metals or conductive organic molecules. The electrodes can be manufactured to virtually any 2-dimensional or 3-dimensional shape (e.g., discrete lines, pads, planes, spheres, cylinders, etc.).

As used herein and unless otherwise indicated, the term “fixed electrode” is intended to reflect the fact that the electrode is essentially stable and unmovable with respect to the storage medium. That is, the electrode and storage medium are arranged in an essentially fixed geometric relationship with each other. It is of course recognized that the relationship alters somewhat due to expansion and contraction of the medium with thermal changes or due to changes in conformation of the molecules comprising the electrode and/or the storage medium. Nevertheless, the overall spatial arrangement remains essentially invariant. In a preferred embodiment this term is intended to exclude systems in which the electrode is a movable “probe” (e.g., a writing or recording “head,” an atomic force microscope (AFM) tip, a scanning tunneling microscope (STM) tip, etc.).

As used herein and unless otherwise indicated, the term “linker” is a molecule used to couple two different molecules, two subunits of a molecule, or a molecule to a substrate.

As used herein and unless otherwise indicated, a metal is designated by “M” or “Mⁿ,” where n is an integer, it is recognized that the metal may be associated with a counter ion.

As used herein and unless otherwise indicated, the term “memory element,” “memory cell,” or “storage cell” refer to an electrochemical cell that can be used for the storage of information. Preferred “storage cells” are discrete regions of storage medium addressed by at least one and preferably by two electrodes (e.g., a working electrode and a reference electrode). The storage cells can be individually addressed (e.g., a unique electrode is associated with each memory element) or, particularly where the oxidation states of different memory elements are distinguishable, multiple memory elements can be addressed by a single electrode. The memory element can optionally include a dielectric (e.g., a dielectric impregnated with counter ions).

As used herein and unless otherwise indicated, the term “multiple oxidation states” means more than one oxidation state. In preferred embodiments, the oxidation states may reflect the gain of electrons (reduction) or the loss of electrons (oxidation).

As used herein and unless otherwise indicated, the term “multiporphyrin array” refers to a discrete number of two or more covalently-linked porphyrinic macrocycles. The multiporphyrin arrays can be linear, cyclic, or branched.

As used herein and unless otherwise indicated, the terms “molecules of the invention,” “self-contained charge storage units” and “self-contained charge storage molecules” (sometimes abbreviated herein as “charge storage units” or “CSUs”) are used interchangably and refer to a molecule possessing an oxidizable functional species, a reducible functional species, and an ionic conducting electrolyte.

As used herein and unless otherwise indicated, the term “output of an integrated circuit” refers to a voltage or signal produced by one or more integrated circuit(s) and/or one or more components of an integrated circuit.

The terms “redox-active molecule (ReAM)” refer to a molecule or component of a molecule that is capable of being oxidized or reduced, e.g., by the application of a suitable voltage. That is, both the oxidizable functional species (Ox) and the reducible functional species (ReS) are both ReAMs. The ReAMs are chosen to be relative, that is, at any particular voltage, the CSUs of the invention contain a ReAM that is in a reducible form and a ReAM in an oxidizable form. As described below, ReAMs can include, but are not limited to macrocycles including porphyrin and porphyrin derivatives, as well as non-macrocyclic compounds, and includes sandwich compounds, e.g. as described herein. In certain embodiments, ReAMs can comprise multiple subunits, for example, in the case of dyads or triads. In general, as described below, there are several types of ReAMs useful in the present invention, all based on polydentate proligands, including macrocyclic and non-macrocyclic moieties. A number of suitable proligands and complexes, as well as suitable substituents, are outlined in U.S. Pat. Nos. 6,212,093; 6,728,129; 6,451,942; 6,777,516; 6,381,169; 6,208,553; 6,657,884; 6,272,038; 6,484,394; and U.S. Ser. Nos. 10/040,059; 10/682,868; 10/445,977; 10/834,630; 10/135,220; 10/723,315; 10/456,321; 10/376,865; all of which are expressly incorporated by reference, in particular for the structures and descriptions thereof depicted therein.

Suitable proligands fall into two categories: ligands which use nitrogen, oxygen, sulfur, carbon or phosphorus atoms (depending on the metal ion) as the coordination atoms (generally referred to in the literature as sigma (σ) donors) and organometallic ligands such as metallocene ligands (generally referred to in the literature as pi (π) donors, and depicted herein as Lm).

In addition, a single ReAM may have two or more redox active subunits (to be distinguished from a CSU with two ReAMs). For example, as shown in Compound 102, there are two redox active subunits, a porphyrin (shown with a Zn metal) and four ferrocenes contained within a single functional species. Similarly, sandwich coordination compounds are considered a single ReAM. This is to be distinguished from the case where these ReAMs are polymerized as monomers. In addition, the metal ions/complexes of the invention may be associated with a counterion, not generally depicted herein.

In one embodiment, the ReAM is a macrocyclic ligand, which includes both macrocyclic proligands and macrocyclic complexes. By “macrocyclic proligand” herein is meant a cyclic compound which contain donor atoms (sometimes referred to herein as “coordination atoms”) oriented so that they can bind to a metal ion and which are large enough to encircle the metal atom. In general, the donor atoms are heteroatoms including, but not limited to, nitrogen, oxygen and sulfur, with the former being especially preferred. However, as will be appreciated by those in the art, different metal ions bind preferentially to different heteroatoms, and thus the heteroatoms used can depend on the desired metal ion. In addition, in some embodiments, a single macrocycle can contain heteroatoms of different types.

A “macrocyclic complex” is a macrocyclic proligand with at least one metal ion; in some embodiments the macrocyclic complex comprises a single metal ion, although as described below, polynucleate complexes, including polynucleate macrocyclic complexes, are also contemplated.

A wide variety of macrocyclic ligands find use in the present invention, including those that are electronically conjugated and those that may not be; however, the macrocyclic ligands of the invention preferably have at least one, and preferably two or more oxidation states, with 4, 6 and 8 oxidation states being of particular significance.

A broad schematic of suitable macrocyclic ligands are shown and described in FIGS. 11 and 14 of U.S. Publication No. 2007/0108438, all of which is incorporated by reference herein in addition to FIGS. 11 and 14. In this embodiment, roughly based on porphyrins, a 16 member ring (when the —X-moiety contains a single atom, either carbon or a heteroatom), 17 membered rings (where one of the —X-moieties contains two skeletal atoms), 18 membered rings (where two of the —X-moieties contains two skeletal atoms), 19 membered rings (where three of the —X-moieties contains two skeletal atoms) or 20 membered rings (where all four of the —X-moieties contains two skeletal atoms), are all contemplated. Each —X-group is independently selected. The ••••Q•••• moiety, together with the skeletal —C-heteroatom-C (with either single or double bonds independently connecting the carbons and heteroatom) for 5 or 6 membered rings that are optionally substituted with 1 or 2 (in the case of 5 membered rings) or 1, 2, or 3 (in the case of 6 membered rings) with independently selected R2 groups. In some embodiments, the rings, bonds and substitutents are chosen to result in the compound being electronically conjugated, and at a minimum to have at least two oxidation states.

In some embodiments, the macrocyclic ligands of the invention are selected from the group consisting of porphyrins (particularly porphyrin derivatives as defined below), and cyclen derivatives.

### Porphyrins

A particularly preferred subset of macrocycles suitable in the invention are porphyrins, including porphyrin derivatives. Such derivatives include porphyrins with extra rings ortho-fused, or ortho-perifused, to the porphyrin nucleus, porphyrins having a replacement of one or more carbon atoms of the porphyrin ring by an atom of another element (skeletal replacement), derivatives having a replacement of a nitrogen atom of the porphyrin ring by an atom of another element (skeletal replacement of nitrogen), derivatives having substituents other than hydrogen located at the peripheral (meso-, (3- or core atoms of the porphyrin, derivatives with saturation of one or more bonds of the porphyrin (hydroporphyrins, e.g., chlorins, bacteriochlorins, isobacteriochlorins, decahydroporphyrins, corphins, pyrrocorphins, etc.), derivatives having one or more atoms, including pyrrolic and pyrromethenyl units, inserted in the porphyrin ring (expanded porphyrins), derivatives having one or more groups removed from the porphyrin ring (contracted porphyrins, e.g., corrin, corrole) and combinations of the foregoing derivatives (e.g. phthalocyanines, sub-phthalocyanines, and porphyrin isomers). Additional suitable porphyrin derivatives include, but are not limited to the chlorophyll group, including etiophyllin, pyrroporphyrin, rhodoporphyrin, phylloporphyrin, phylloerythrin, chlorophyll a and b, as well as the hemoglobin group, including deuteroporphyrin, deuterohemin, hemin, hematin, protoporphyrin, mesohemin, hematoporphyrin mesoporphyrin, coproporphyrin, uruporphyrin and turacin, and the series of tetraarylazadipyrromethines.

As is true for the compounds outlined herein, and as will be appreciated by those in the art, each unsaturated position, whether carbon or heteroatom, can include one or more substitution groups as defined herein, depending on the desired valency of the system.

In one preferred embodiment, the redox-active molecule may be a metallocene, which can be substituted at any appropriate position, using R groups independently selected herein. A metallocene which finds particular use in the invention includes ferrocene and its derivatives. In this embodiment, preferred substituents include, but are not limited to, 4-chlorophenyl, 3-acetamidophenyl, 2,4-dichloro-4-trifluoromethyl. Preferred substituents provide a redox potential range of less than about 2 volts.

It will be appreciated that the oxidation potentials of the members of the series can be routinely altered by changing the metal (M) or the substituents.

Control over the hole-storage and hole-hopping properties of the redox-active units of the redox-active molecules used in the memory devices of the present invention allows fine control over the architecture of the memory device.

Such control is exercised through synthetic design. The hole-storage properties depend on the oxidation potential of the redox-active units or subunits that are themselves or are used to assemble the storage media used in the devices of this invention. The hole-storage properties and redox potential can be tuned with precision by choice of base molecule(s), associated metals and peripheral substituents (Yang et al. (1999) J. Porphyrins Phthalocyanines, 3: 117-147), the disclosure of which is herein incorporated by this reference.

For example, in the case of porphyrins, Mg porphyrins are more easily oxidized than Zn porphyrins, and electron withdrawing or electron releasing aryl groups can modulate the oxidation properties in predictable ways. Hole-hopping occurs among isoenergetic porphyrins in a nanostructure and is mediated via the covalent linker joining the porphyrins (Seth et al. (1994) J. Am. Chem. Soc., 116: 10578-10592, Seth et al (1996) J. Am. Chem. Soc., 118: 11194-11207, Strachan et al. (1997) J. Am. Chem. Soc., 119: 11191-11201; Li et al. (1997) J. Mater. Chem., 7: 1245-1262, Strachan et al. (1998) Inorg. Chem., 37: 1191-1201, Yang et al. (1999) J. Am. Chem. Soc., 121: 4008-4018), the disclosures of which are herein specifically incorporated by this reference in their entirety. The design of compounds with predicted redox potentials is well known to those of ordinary skill in the art. In general, the oxidation potentials of redox-active units or subunits are well known to those of skill in the art and can be looked up (see, e.g., Handbook of Electrochemistry of the Elements). Moreover, in general, the effects of various substituents on the redox potentials of a molecule are generally additive. Thus, a theoretical oxidation potential can be readily predicted for any potential data storage molecule. The actual oxidation potential, particularly the oxidation potential of the information storage molecule(s) or the information storage medium can be measured according to standard methods. Typically the oxidation potential is predicted by comparison of the experimentally determined oxidation potential of a base molecule and that of a base molecule bearing one substituent in order to determine the shift in potential due to that particular substituent. The sum of such substituent-dependent potential shifts for the respective substituents then gives the predicted oxidation potential.

The suitability of particular redox-active molecules for use in the methods of this invention can readily be determined. The molecule(s) of interest are simply polymerized and coupled to a surface (e.g., a hydrogen passivated surface) according to the methods of this invention. Then sinusoidal voltammetry can be performed (e.g., as described herein or in U.S. Pat. Nos. 6,272,038; 6,212,093; and 6,208,553, PCT Publication WO 01/03126, or by (Roth et al. (2000) Vac. Sci. Technol. B 18:2359-2364; Roth et al. (2003) J. Am. Chem. Soc. 125:505-517) to evaluate 1) whether or not the molecule(s) coupled to the surface, 2) the degree of coverage (coupling); 3) whether or not the molecule(s) are degraded during the coupling procedure, and 4) the stability of the molecule(s) to multiple read/write operations.

In addition, included within the definition of “porphyrin” are porphyrin complexes, which comprise the porphyrin proligand and at least one metal ion. Suitable metals for the porphyrin compounds will depend on the heteroatoms used as coordination atoms, but in general are selected from transition metal ions. The term “transition metals” as used herein typically refers to the 38 elements in groups 3 through 12 of the periodic table. Typically transition metals are characterized by the fact that their valence electrons, or the electrons they use to combine with other elements, are present in more than one shell and consequently often exhibit several common oxidation states. In certain embodiments, the transition metals of this invention include, but are not limited to one or more of scandium, titanium, vanadium, chromium, manganese, iron, cobalt, nickel, copper, zinc, yttrium, zirconium, niobium, molybdenum, technetium, ruthenium, rhodium, palladium, silver, cadmium, hafnium, tantalum, tungsten, rhenium, osmium, iridium, platinum, gold, mercury, rutherfordium, and/or oxides, and/or nitrides, and/or alloys, and/or mixtures thereof.

### Other Macrocycles

There are a number of macrocycles based on cyclen derivatives based on cyclen/cyclam derivatives which can include skeletal expansion by the inclusion of independently selected carbons or heteroatoms, having the general formula:

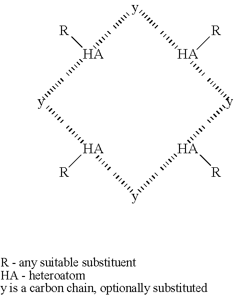

In some embodiments, at least one R group is a redox active subunit, preferably electronically conjugated to the metal. In some embodiments, including when at least one R group is a redox active subunit, two or more neighboring R groups form cyclo or an aryl group. In addition, metals can optionally be present as described herein. In some embodiments, at least one R group is a redox active subunit, preferably electronically conjugated to the metal. In some embodiments, including when at least one R group is a redox active subunit, two or more neighboring R2 groups form cyclo or an aryl group.

Furthermore, in some embodiments, macrocyclic complexes relying organometallic ligands are used. In addition to purely organic compounds for use as redox moieties, and various transition metal coordination complexes with δ-bonded organic ligand with donor atoms as heterocyclic or exocyclic substituents, there is available a wide variety of transition metal organometallic compounds with π-bonded organic ligands (see Advanced Inorganic Chemistry, 5th Ed., Cotton & Wilkinson, John Wiley & Sons, 1988, chapter 26; Organometallics, A Concise Introduction, Elschenbroich et al., 2nd Ed., 1992, VCH; and Comprehensive Organometallic Chemistry II, A Review of the Literature 1982-1994, Abel et al. Ed., Vol. 7, chapters 7, 8, 10 & 11, Pergamon Press, hereby expressly incorporated by reference). Such organometallic ligands include cyclic aromatic compounds such as the cyclopentadienide ion [C5H5(−1)] and various ring substituted and ring fused derivatives, such as the indenylide (−1) ion, that yield a class of bis(cyclopentadieyl)metal compounds, (i.e. the metallocenes); see for example Robins et al., J. Am. Chem. Soc. 104:1882-1893 (1982); and Gassman et al., J. Am. Chem. Soc. 108:4228-4229 (1986), incorporated by reference. Of these, ferrocene [(C5H5)2Fe] and its derivatives are prototypical examples which have been used in a wide variety of chemical (Connelly et al., Chem. Rev. 96:877-910 (1996), incorporated by reference) and electrochemical (Geiger et al., Advances in Organometallic Chemistry 23:1-93; and Geiger et al., Advances in Organometallic Chemistry 24:87, incorporated by reference) electron transfer or “redox” reactions. Metallocene derivatives of a variety of the first, second and third row transition metals are useful as redox moieties (and redox subunits). Other potentially suitable organometallic ligands include cyclic arenes such as benzene, to yield bis(arene)metal compounds and their ring substituted and ring fused derivatives, of which bis(benzene)chromium is a prototypical example, Other acyclic π-bonded ligands such as the allyl(−1) ion, or butadiene yield potentially suitable organometallic compounds, and all such ligands, in conjunction with other π-bonded and δ-bonded ligands constitute the general class of organometallic compounds in which there is a metal to carbon bond. Electrochemical studies of various dimers and oligomers of such compounds with bridging organic ligands, and additional non-bridging ligands, as well as with and without metal-metal bonds are all useful.

When one or more of the co-ligands is an organometallic ligand, the ligand is generally attached via one of the carbon atoms of the organometallic ligand, although attachment may be via other atoms for heterocyclic ligands. Preferred organometallic ligands include metallocene ligands, including substituted derivatives and the metalloceneophanes (see page 1174 of Cotton and Wilkenson, supra). For example, derivatives of metallocene ligands such as methylcyclopentadienyl, with multiple methyl groups being preferred, such as pentamethylcyclopentadienyl, can be used to increase the stability of the metallocene. In some embodiments, the metallocene is derivatized with one or more substituents as outlined herein, particularly to alter the redox potential of the subunit or moiety.

As described herein, any combination of ligands may be used. Preferred combinations include: a) all ligands are nitrogen donating ligands; b) all ligands are organometallic ligands.

### Sandwich Coordination Complexes

In some embodiments, the ReAMs are sandwich coordination complexes. The terms “sandwich coordination compound” or “sandwich coordination complex” refer to a compound of the formula L-Mn-L, where each L is a heterocyclic ligand (as described below), each M is a metal, n is 2 or more, most preferably 2 or 3, and each metal is positioned between a pair of ligands and bonded to one or more hetero atom (and typically a plurality of hetero atoms, e.g., 2, 3, 4, 5) in each ligand (depending upon the oxidation state of the metal). Thus sandwich coordination compounds are not organometallic compounds such as ferrocene, in which the metal is bonded to carbon atoms. The ligands in the sandwich coordination compound are generally arranged in a stacked orientation (i.e., are generally cofacially oriented and axially aligned with one another, although they may or may not be rotated about that axis with respect to one another) (see, e.g., Ng and Jiang (1997) Chemical Society Reviews 26: 433-442) incorporated by reference. Sandwich coordination complexes include, but are not limited to “double-decker sandwich coordination compound” and “triple-decker sandwich coordination compounds”. The synthesis and use of sandwich coordination compounds is described in detail in U.S. Pat. Nos. 6,212,093; 6,451,942; 6,777,516; and polymerization of these molecules is described in U.S. Publication No. 20070123618, all of which are included herein, particularly the individual substitutent groups that find use in both sandwich complexes and the “single” macrocycle” complexes.

The term “double-decker sandwich coordination compound” refers to a sandwich coordination compound as described above where n is 2, thus having the formula L′-M′-LZ, wherein each of L1 and LZ may be the same or different (see, e.g., Jiang et al. (1999) J. Porphyrins Phthalocyanines 3: 322-328) and U.S. Pat. Nos. 6,212,093; 6,451,942; 6,777,516; and polymerization of these molecules is described in U.S. Publication No. 20070123618, hereby incorporated by reference in its entirety.

The term “triple-decker sandwich coordination compound” refers to a sandwich coordination compound as described above where n is 3, thus having the formula L′-M′LZ-MZ-L3, wherein each of L1, LZ and L3 may be the same or different, and M1 and MZ may be the same or different (see, e.g., Arnold et al. (1999) Chemistry Letters 483-484), and U.S. Pat. Nos. 6,212,093; 6,451,942; 6,777,516; and polymerization of these molecules is described in U.S. Publication No. 20070123618, hereby incorporated by reference in their entirety.

In addition, polymers of these sandwich compounds are also of use; this includes “dyads” and “triads” as described in U.S. Pat. Nos. 6,212,093; 6,451,942; 6,777,516; and polymerization of these molecules is described in U.S. Publication No. 20070123618, incorporated by reference.

### Non-Macrocyclic Proligands and Complexes

As a general rule, ReAMs comprising non-macrocyclic chelators are bound to metal ions to form non-macrocyclic chelate compounds, since the presence of the metal allows for multiple proligands to bind together to give multiple oxidation states.

In some embodiments, nitrogen donating proligands are used. Suitable nitrogen donating proligands are well known in the art and include, but are not limited to, NH2; NHR; NRR′; pyridine; pyrazine; isonicotinamide; imidazole; bipyridine and substituted derivatives of bipyridine; terpyridine and substituted derivatives; phenanthrolines, particularly 1,10-phenanthroline (abbreviated phen) and substituted derivatives of phenanthrolines such as 4,7-dimethylphenanthroline and dipyridol[3,2-a:2′,3′-c]phenazine (abbreviated dppz); dipyridophenazine; 1,4,5,8,9,12-hexaazatriphenylene (abbreviated hat); 9,10-phenanthrenequinone diimine (abbreviated phi); 1,4,5,8-tetraazaphenanthrene (abbreviated tap); 1,4,8,11-tetra-azacyclotetradecane (abbreviated cyclam) and isocyanide. Substituted derivatives, including fused derivatives, may also be used. It should be noted that macrocylic ligands that do not coordinatively saturate the metal ion, and which require the addition of another proligand, are considered non-macrocyclic for this purpose. As will be appreciated by those in the art, it is possible to covalent attach a number of “non-macrocyclic” ligands to form a coordinatively saturated compound, but that is lacking a cyclic skeleton.

Suitable sigma donating ligands using carbon, oxygen, sulfur and phosphorus are known in the art. For example, suitable sigma carbon donors are found in Cotton and Wilkenson, Advanced Organic Chemistry, 5th Edition, John Wiley & Sons, 1988, hereby incorporated by reference; see page 38, for example. Similarly, suitable oxygen ligands include crown ethers, water and others known in the art. Phosphines and substituted phosphines are also suitable; see page 38 of Cotton and Wilkenson.

The oxygen, sulfur, phosphorus and nitrogen-donating ligands are attached in such a manner as to allow the heteroatoms to serve as coordination atoms.

### Polynucleating Proligands and Complexes

In addition, some embodiments utilize polydentate ligands that are polynucleating ligands, e.g. they are capable of binding more than one metal ion. These may be macrocyclic or non-macrocyclic.

A number of suitable proligands and complexes, as well as suitable substituents, are outlined in U.S. Pat. Nos. 6,212,093; 6,728,129; 6,451,942; 6,777,516; 6,381,169; 6,208,553; 6,657,884; 6,272,038; 6,484,394; and U.S. Ser. Nos. 10/040,059; 10/682,868; 10/445,977; 10/834,630; 10/135,220; 10/723,315; 10/456,321; 10/376,865; all of which are expressly incorporated by reference, in particular for the structures and descriptions thereof depicted therein.

As used herein and unless otherwise indicated, the term “present on a single plane,” when used in reference to a memory device of this invention refers to the fact that the component(s) (e.g. storage medium, electrode(s), etc.) in question are present on the same physical plane in the device (e.g. are present on a single lamina). Components that are on the same plane can typically be fabricated at the same time, e.g., in a single operation. Thus, for example, all of the electrodes on a single plane can typically be applied in a single (e.g., sputtering) step (assuming they are all of the same material).

As used herein and unless otherwise indicated, a potentiometric device is a device capable of measuring potential across an interface that results from a difference in the equilibrium concentrations of redox molecules in an electrochemical cell.

As used herein and unless otherwise indicated, the term “oxidation” refers to the loss of one or more electrons in an element, compound, or chemical substituent/subunit. In an oxidation reaction, electrons are lost by atoms of the element(s) involved in the reaction. The charge on these atoms must then become more positive. The electrons are lost from the species undergoing oxidation and so electrons appear as products in an oxidation reaction. An oxidation taking place in the reaction Fe²⁺(aq)→Fe³⁺(aq)+e⁻ because electrons are lost from the species being oxidized, Fe²⁺(aq), despite the apparent production of electrons as “free” entities in oxidation reactions. Conversely the term reduction refers to the gain of one or more electrons by an element, compound, or chemical substituent/subunit.

As used herein and unless otherwise indicated, the term “oxidation state” refers to the electrically neutral state or to the state produced by the gain or loss of electrons to an element, compound, or chemical substituent/subunit. In a preferred embodiment, the term “oxidation state” refers to states including the neutral state and any state other than a neutral state caused by the gain or loss of electrons (reduction or oxidation).

As used herein and unless otherwise indicated, the term “read” or “interrogate” refer to the determination of the oxidation state(s) of one or more molecules (e.g. molecules comprising a storage medium).

As used herein and unless otherwise indicated, the term “redox-active unit” or “redox-active subunit” refers to a molecule or component of a molecule that is capable of being oxidized or reduced by the application of a suitable voltage.

As used herein and unless otherwise indicated, the terms “read” or “interrogate” refer to the determination of the oxidation state(s) of one or more molecules (e.g. molecules comprising a storage medium).

As used herein and unless otherwise indicated, the term “refresh” when used in reference to a storage molecule or to a storage medium refers to the application of a voltage to the storage molecule or storage medium to re-set the oxidation state of that storage molecule or storage medium to a predetermined state (e.g., the oxidation state the storage molecule or storage medium was in immediately prior to a read).

As used herein and unless otherwise indicated, the term “reference electrode” is used to refer to one or more electrodes that provide a reference (e.g., a particular reference voltage) for measurements recorded from the working electrode. In preferred embodiments, the reference electrodes in a memory device of this invention are at the same potential although in some embodiments this need not be the case.

As used herein and unless otherwise indicated, a “sinusoidal voltammeter” is a voltammetric device capable of determining the frequency domain properties of an electrochemical cell.

As used herein and unless otherwise indicated, the term “storage density” refers to the number of bits per volume and/or bits per molecule that can be stored. When the storage medium is said to have a storage density greater than one bit per molecule, this refers to the fact that a storage medium preferably comprises molecules wherein a single molecule is capable of storing at least one bit of information.

As used herein and unless otherwise indicated, the term “storage location” refers to a discrete domain or area in which a storage medium is disposed. When addressed with one or more electrodes, the storage location may form a storage cell. However if two storage locations contain the same storage media so that they have essentially the same oxidation states, and both storage locations are commonly addressed, they may form one functional storage cell.

As used herein and unless otherwise indicated, the term “storage medium” refers to a composition comprising a storage molecule of the invention, preferably bonded to a substrate.

A substrate is a, preferably solid, material suitable for the attachment of one or more molecules. Substrates can be formed of materials including, but not limited to glass, plastic, silicon, minerals (e.g., quartz), semiconducting materials, ceramics, metals, etc.

Many of the compounds described herein utilize substituents, generally depicted herein as “R.” Suitable R groups include, but are not limited to, hydrogen, alkyl, alcohol, aryl, amino, amido, nitro, ethers, esters, aldehydes, sulfonyl, silicon moieties, halogens, cyano, acyl, sulfur containing moieties, phosphorus containing moieties, amido, imido, carbamoyl, linkers, attachment moieties, ReAMs and other subunits. It should be noted that some positions may allow two substitution groups, R and R′, in which case the R and R′ groups may be either the same or different, and it is generally preferred that one of the substitution groups be hydrogen. In some embodiments, the R groups are as defined and depicted in the figures and the text from U.S A number of suitable proligands and complexes, as well as suitable substituents, are outlined in U.S. Pat. Nos. 6,212,093; 6,728,129; 6,451,942; 6,777,516; 6,381,169; 6,208,553; 6,657,884; 6,272,038; 6,484,394; and U.S. Ser. Nos. 10/040,059; 10/682,868; 10/445,977; 10/834,630; 10/135,220; 10/723,315; 10/456,321; 10/376,865; all of which are expressly incorporated by reference, in particular for the structures and descriptions thereof depicted therein, hereby expressly incorporated as substitutent embodiments, both for the particular macrocycle the substituents are depicted within and for further substituted derivatives.

As used herein and unless otherwise indicated, the term “sulfoxyl” refers to a group of composition RS(O)— where R is some alkyl, aryl, cycloalkyl, perfluoroalkyl, or perfluoroaryl group. Examples include, but are not limited to methylsulfoxyl, phenylsulfoxyl, etc.

As used herein and unless otherwise indicated, the term “sulfonyl” refers to a group of composition RSO₂, where R is some alkyl, aryl, cycloalkyl, perfluoroalkyl, or perfluoroaryl group. Examples include, but are not limited to methylsulfonyl, phenylsulfonyl, p-toluenesulfonyl, etc.

As used herein and unless otherwise indicated, the term “subunit” refers to a redox-active component of a molecule.

As used herein and unless otherwise indicated, the term “thiocyanato” refers to the —SCN group.

As used herein and unless otherwise indicated, the term “voltammetric device” is a device capable of measuring the current produced in an electrochemical cell as a result of the application of a voltage or change in voltage.

As used herein and unless otherwise indicated, a voltage source is any source (e.g. molecule, device, circuit, etc.) capable of applying a voltage to a target (e.g., an electrode).

As used herein and unless otherwise indicated, the term “working electrode” is used to refer to one or more electrodes that are used to set or read the state of a storage medium and/or storage molecule.

### 5.2. Compositions of the Invention

The invention encompasses novel self-contained charge storage units (e.g., molecules) that contain three functionalities and thus greatly simplify the manufacturing and reproducibility of devices containing these compounds. The CSU molecules comprise:
(a) one or more reducible functional species (ReS);
(b) one or more oxidizable functional species (Ox); and
(c) one or more ion conducting moiety (IC).

In an illustrative embodiment of the self-contained charge storage molecule each of the one or more reducible species components (ReS), the one or more oxidizable species components (Ox), and the one or more ion conducting moiety components (IC) are chemically attached, preferably covalently.

In another illustrative embodiment, one or more reducible species are attached to one or more ion conducting moieties and one or more ion conducting moieties are attached to one or more oxidizable species components in the following manner:

(ReS)ₗ-(IC)ₘ-(Ox)ₙ.

In this embodiment, l, m and n are each integers. As is outlined herein, there may be more than one of a particular species, and they may be attached in any orientation.

That is, the arrangements of the functional species relative to each other within the self-contained charge storage unit has no prerequisite and can be chosen based on the ease of preparation, physical characteristics of the bulk materials, chemical compatibility with the electrode materials and any other attribute that allows the self-contained charge storage unit to function. Preferably, the molecular linkages within the self-contained charge storage unit may be chosen so as to afford desired properties, for example, thermo-mechanical stability, thermo-chemical stability, operation speed, charge retention time, and other electrochemical properties. In a preferred embodiment, the functional species of the self-contained charge storage unit are linked to each other chemically, for example, by an ionic or covalent bond, to avoid irreversible phase separation. Preferably, the functional species are covalently linked.

In another illustrative embodiment, the ionic components of the self-contained charge storage unit should lack reactivity toward the oxidized and neutral forms of the oxidizable species (Ox) and reduced and neutral forms of the reducible species (ReS).

In another embodiment, the material constituting the self-contained charge storage unit should be applied in such a way that molecular reorganization and/or reorientation can occur to the extent necessary for redox recycling of components (ReS) and (Ox).

In another embodiment, the fabrication of stable and reliable molecular devices requires an attachment (e.g., covalent) of the self-contained charge storage molecule at any site (e.g., a covalent bond from the reducible functional species, the oxidizable functional species, or the ionic conducting moiety) to a device substrate (e.g., silicon) or electrode. As will be appreciated by those in the art, the attachment chemistry will depend on the composition of the surface to which attachment is desired. Generally, the attachment of molecules to silicon can be accomplished through covalent linkage chemistries involving formation of Si—C or Si—O bonds. Preferably, the stability of the bond formed between the substrate and the molecules will dictate the thermal and electrical stability of the self-assembled monolayer, as described herein.

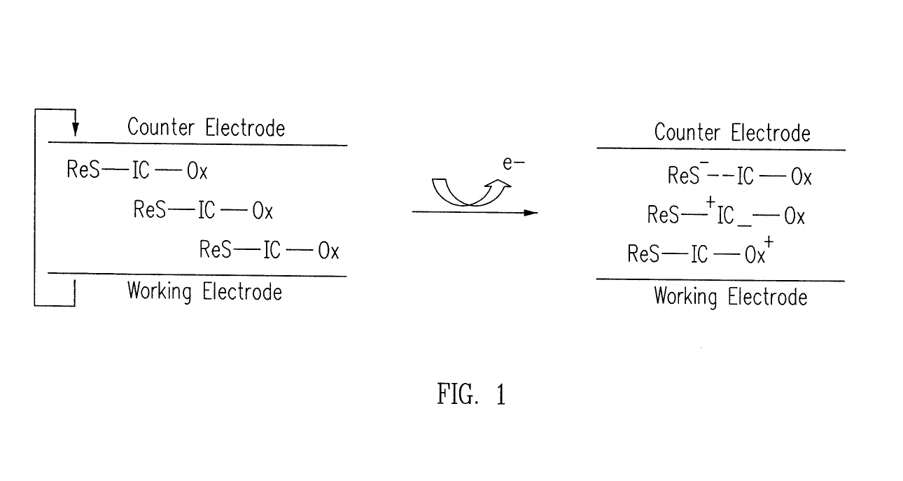

FIG. 1 illustrates the operation/functionality of a self-contained charge storage unit in accordance with some embodiments of the present invention. In the unbiased/uncharged state, the conformation of the charge storage molecules may be random or ordered to some degree, depending on the methods of deposition of the molecules and electrode materials. In order for the redox activity to occur the molecules should be either in an acceptable environment or be able to reorganize such that charge stabilization can occur (i.e., ion pairing of appropriate counter ions of the ion conducting species (IC) must diffuse in order to pair with developing charges of the oxidizable species (Ox) and the reducible species (ReS)).

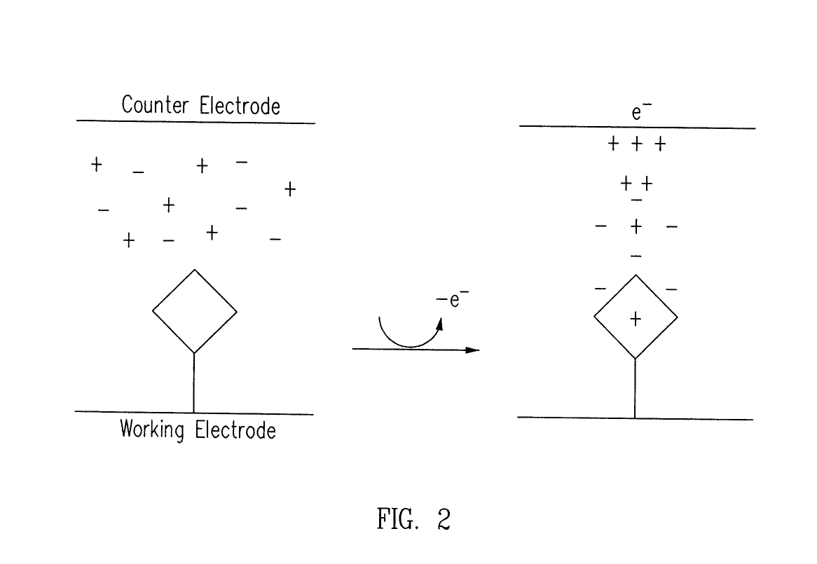

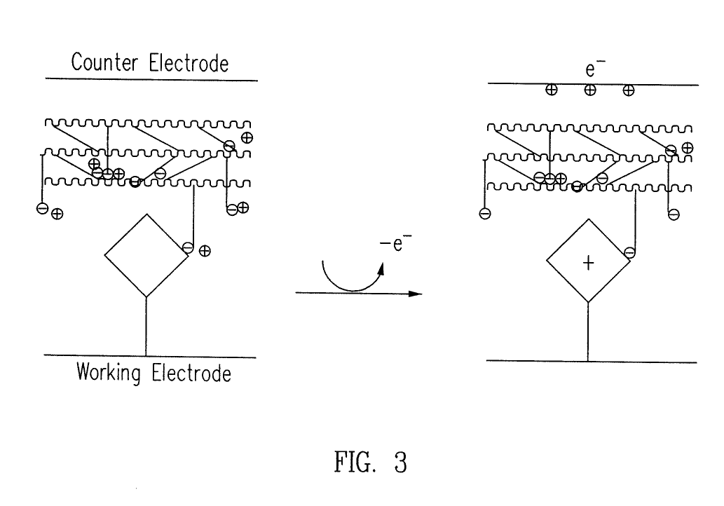

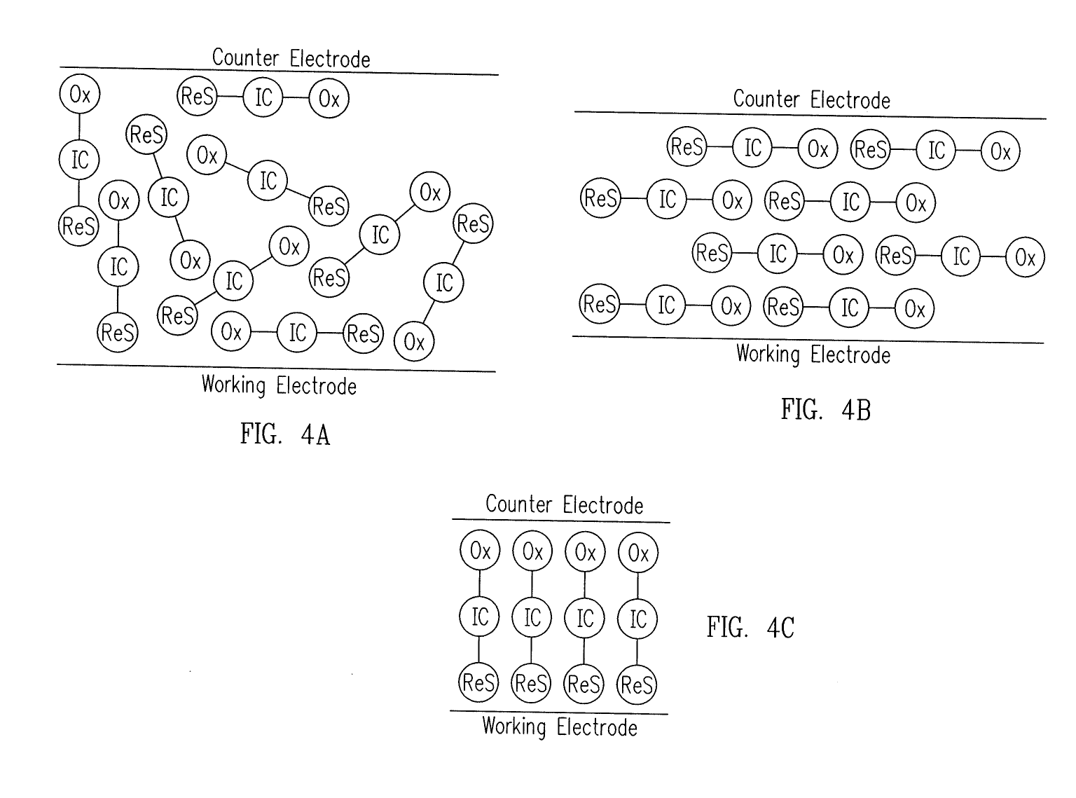

FIG. 2 illustrates molecular memory in a liquid electrolyte system in accordance with some embodiments of the present invention. In a liquid electrolyte system the electrolyte structure is unperturbed and is an inherent property of the electrolyte. Upon molecule oxidation, kinetic electrolyte “re-structuring” dominates the “observed” CV characteristics under rapid cycling conditions, while thermodynamic “restructuring” dominates the observed CV characteristics under slow cycling conditions. FIG. 3 shows molecular memory in a solid system in accordance with other embodiments of the present invention, and illustrates one example of how the ionic liquid electrolyte reorients as a result of a “write step,” meaning oxidation of the Ox species and reduction of the ReS species. FIGS. 4A to 4C illustrate molecular memory in three different orientations of the self-contained charge storage unit (CSU) molecules or moieties, according to various embodiments of the present invention. Of particular advantage, the CSU molecule remains functionally capable, regardless of the orientation of the CSU molecule.

5.2.1. The Oxidizable Functional Species

The oxidizable functional species of the self-contained charge storage unit is preferably held in close proximity to an electrically, positively-biased electrode, for example, the working electrode. Preferably, the oxidizable functional species is chemically bound to an electrically, positively-biased electrode; most preferably the oxidizable functional species is covalently bound to the electrically, positively-biased electrode.

Generally, the oxidizable functional species that can be utilized as the oxidizable functional species of the self-contained charge storage unit can be any stable molecular species capable of being oxidized within a suitable electrochemical window, generally described herein as a ReAM. Preferably, the oxidizable functional species includes those that are electronically conjugated and those that may not be; however, the oxidizable functional species of the invention preferably have at least one, and preferably more oxidation states. In a particular embodiment, oxidizable functional species with 4, 6 and 8 oxidation states are preferred.

In an illustrative embodiment, the oxidizable functional species can be a macrocyclic component that has a structure of the general formula:

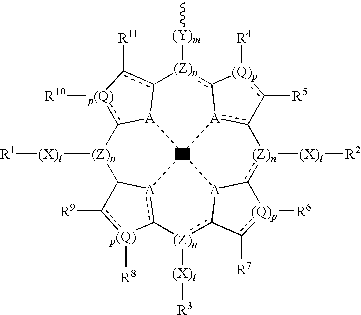

wherein:

each A is independently N, O, S, Se, Te, CH, or CH₂;

M is absent or is a metal including, but not limited to, Zn, Mg, Cd, Hg, Cu, Ag, Au, Ni, Pd, Pt, Co, Rh, Ir, Mn, B, Al, Ga, Pb, and Sn;

each Z is independently N, O, S, Si, Se, Te, CH, or CH₂;

X and Y are each independently a bond or NH, O, Si, S, Se, Te, or CH₂;

R¹, R², and R³ are optional and each independently a substituent group. In some embodiments, they can be independently selected from ReS, an additional ReAM, bipyridyl, bipyridinium, aryl, benzyl, cycloalkyl, alkyl, halogen, alkoxy, alkylthio, perfluoroalkyl, perfluoroaryl, pyridyl, cyano, thiocyanato, nitro, amino, alkylamino, acyl, sulfoxyl, sulfonyl, imido, amido, and carbamoyl;

each R⁴, R⁵, R⁶, R⁷, R⁸, R⁹ and R¹⁰ is optional and independently a substituent group as defined herein. In some embodiments, they can be independently selected from ReS, an additional ReAM, —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂;

each Q is independently a CH or a heteroatom;

each n is independently 0-3;

m is 0-5;

each l is independently 0-5;

each p is independently 0-3; and

[symbol] is an attachment, preferably covalent to the reducible species or the ionic conducting electrolyte. While depicted in this formula as attached via the “Y” group, those of skill in the art will appreciate that attachment can occur at any “R” position as well. In one embodiment, at least one A is nitrogen. In another embodiment, each A is a nitrogen. In another embodiment, at least one A is oxygen. In another embodiment, each A is an oxygen. In another embodiment, at least one A is sulfur. In another embodiment, each A is a sulfur. In another embodiment, at least one A is methylene. In another embodiment, each A is a methylene. In another embodiment, at least one A is selenium. In another embodiment, each A is a selenium. In another embodiment, at least one A is tellurium. In another embodiment, each A is a telerium.

In one embodiment, M is absent. In one embodiment, M is Zn. In another embodiment, M is Mg. In another embodiment, M is Cd. In another embodiment, M is Hg.

In another embodiment, M is Cu. In another embodiment, M is Ag. In another embodiment, M is Au. In another embodiment, M is Ni. In another embodiment, M is Pd. In another embodiment, M is Pt. In another embodiment, M is Co. In another embodiment, M is Rh. In another embodiment, M is Mn. In another embodiment, M is B. In another embodiment, M is Al. In another embodiment, M is Ga. In another embodiment, M is Pb. In another embodiment, M is Sn. In addition, when multiple metals are present (e.g. where the oxidizable species contains multiple metals, or where each of the oxidizable and reducible species contains metals), the metals are independently selected from this list, rendering any combination possible.

In another embodiment, at least one Z is N or NH. In another embodiment, at least one Z is O. In another embodiment, at least one Z is S. In another embodiment, at least one Z is Se. In another embodiment, at least one Z is CH or C. In another embodiment, at least one Z is Te. In another embodiment, at least one Z is Se.

In another embodiment, X is —NH or N. In another embodiment, X is S. In another embodiment, X is Se. In another embodiment, X is Te. In another embodiment, X is CH or CH₂. In another embodiment, Y is —NH or N. In another embodiment, Y is S. In another embodiment, Y is Se. In another embodiment, Y is Te. In another embodiment, Y is CH or CH₂.

In another embodiment, at least one of R¹, R² or R³ is hydrogen. In another embodiment, at least one of R¹, R² or R³ is benzyl. In another embodiment, at least one of R¹, R² or R³ is cycloalkyl. In another embodiment, at least one of R¹, R² or R³ is alkyl. In another embodiment, at least one of R¹, R² or R³ is halogen. In another embodiment, at least one of R¹, R² or R³ is alkoxy. In another embodiment, at least one of R¹, R² or R³ is alkylthio. In another embodiment, at least one of R¹, R² or R³ is perfluoroalkyl. In another embodiment, at least one of R¹, R² or R³ is perfluoroaryl. In another embodiment, at least one of R¹, R² or R³ is pyridyl. In another embodiment, at least one of R¹, R² or R³ is cyano. In another embodiment, at least one of R¹, R² or R³ is thiocyanato. In another embodiment, at least one of R¹, R² or R³ is nitro. In another embodiment, at least one of R¹, R² or R³ is amino. In another embodiment, at least one of R¹, R² or R³ is alkylamino. In another embodiment, at least one of R¹, R² or R³ is acyl. In another embodiment, at least one of R¹, R² or R³ is sulfoxyl. In another embodiment, at least one of R¹, R² or R³ is sulfonyl. In another embodiment, at least one of R¹, R² or R³ is imido. In another embodiment, at least one of R¹, R² or R³ is amido. In another embodiment, at least one of R¹, R² or R³ is carbamoyl.

In another embodiment, at least one of R⁴, R⁵, R⁶, R⁷, R⁸, R⁹, or R¹⁰ is —H. In another embodiment, at least one of R⁴, R⁵, R⁶, R⁷, R⁸, R⁹, or R¹⁰ is —OH. In another embodiment, at least one of R⁴, R⁵, R⁶, R⁷, R⁸, R⁹, or R¹⁰ is —N(C₁-C₁₀)alkyl(C₁-C₁₀)alkyl. In another embodiment, at least one of R⁴, R⁵, R⁶, R⁷, R⁸, R⁹, or R¹⁰ is —(C₁-C₁₀)alkyl. In another embodiment, at least one of R⁴, R⁵, R⁶, R⁷, R⁸, R⁹, or R¹⁰ is —O(C₁-C₁₀)alkyl. In another embodiment, at least one of R⁴, R⁵, R⁶, R⁷, R⁸, R⁹, or R¹⁰ is —O—C(O)(C₁-C₁₀)alkyl. In another embodiment, at least one of R⁴, R⁵, R⁶, R⁷, R⁸, R⁹, or R¹⁰ is —NH(CH₂)ₘ(C₁-C₁₀)alkyl. In another embodiment, at least one of R⁴, R⁵, R⁶, R⁷, R⁸, R⁹, or R¹⁰ is —O—CF₃. In another embodiment, at least one of R⁴, R⁵, R⁶, R⁷, R⁸, R⁹, or R¹⁰ is —O-benzyl. In another embodiment, at least one of R⁴, R⁵, R⁶, R⁷, R⁸, R⁹, or R¹ is —O—(CH₂)ₘCH((C₁-C₁₀)alkyl(C₁-C₁₀)alkyl). In another embodiment, at least one of R⁴, R⁵, R⁶, R⁷, R⁸, R⁹, or R¹⁰ is —(C₂-C₁₀)alkenyl. In another embodiment, at least one of R⁴, R⁵, R⁶, R⁷, R⁸, R⁹, or R¹⁰ is —(C₂-C₁₀)alkynyl. In another embodiment, at least one of R⁴, R⁵, R⁶, R⁷, R⁸, R⁹ or R¹⁰ is —(C₃-C₁₀)cycloalkyl. In another embodiment, at least one of R⁴, R⁵, R⁶, R⁷, R⁸, R⁹, or R¹⁰ is —(C₈-C₁₄)bicycloalkyl. In another embodiment, at least one of R⁴, R⁵, R⁶, R⁷, R⁸, R⁹ or R¹⁰ is —(C₈-C₁₀)cycloalkenyl. In another embodiment, at least one of R⁴, R⁵, R⁶, R⁷, R⁸, R⁹, or R¹⁰ is —O—(C₂-C₁₀)alkenyl. In another embodiment, at least one of R⁴, R⁵, R⁶, R⁸, R⁹, or R¹⁰ is —O—(C₂-C₁₀)alkynyl. In another embodiment, at least one of R⁴, R⁵, R⁶, R⁸, R⁹, or R¹⁰ is —O—(C₃-C₁₀)cycloalkyl. In another embodiment, at least one of R⁴, R⁵, R⁶, R⁷, R⁸, R⁹ or R¹⁰ is —O—(C₈-C₁₄)bicycloalkyl. In another embodiment, at least one of R⁴, R⁵, R⁶, R⁷, R⁸, R⁹, or R¹⁰ is —O—(C₅-C₁₀)cycloalkenyl. In another embodiment, at least one of R⁴, R⁵, R⁶, R⁷, R⁸, R⁹ or R¹⁰ is —O-heteroaryl. In another embodiment, at least one of R⁴, R⁵, R⁶R⁷R⁸R⁹ or R¹⁰ is -heteroaryl. In another embodiment, at least one of R⁴, R⁵, R⁶, R⁷, R⁸, R⁹, or R¹⁰ is -aryl. In another embodiment, at least one of R⁴, R⁵, R⁶, R⁷, R⁸, R⁹, or R¹⁰ is —(C₃-C₁₀)heterocyclealkyl. In another embodiment, at least one of R⁴, R⁵, R⁶, R⁷, R⁸, R⁹ or R¹⁰ is —O—(CH₂)ₙ-aryl. In another embodiment, at least one of R⁴, R⁵, R⁶, R⁷, R⁹, or R¹⁰ is —O—(C₃-C₁₀)heterocycloalkyl. In another embodiment, at least one of R⁴, R⁵, R⁶, R⁷, R⁸, R⁹ or R¹⁰ is —NHC(O)(C₁-C₁₀)alkyl. In another embodiment, at least one of R⁴, R⁵, R⁶, R⁷, R⁸, R⁹, or R¹⁰ is —NHC(O)NH(C₁-C₁₀)alkyl. In another embodiment, at least one of R⁴, R⁵, R⁶, R⁷, R⁸, R⁹, or R¹⁰ is —NH(aryl). In another embodiment, at least one of R⁴, R⁵, R⁶, R⁷, R⁸, R⁹, or R¹⁰ is —N═C(aryl). In another embodiment, at least one of R⁴, R⁵, R⁶, R⁷, R⁸, R⁹, or R¹⁰ is —SO₂NH₂.

In one illustrative embodiment, the oxidizable functional species is a macrocyclic component, which includes cyclic compounds, which contain donor atoms referred to herein as “coordination atoms” oriented so that they can bind to a metal ion and which are large enough to encircle the metal atom. In general, the donor atoms are heteroatoms including, but not limited to, nitrogen, oxygen and sulfur, with nitrogen being especially preferred. However, as will be appreciated by those in the art, different metal ions bind preferentially to different heteroatoms, and thus the heteroatoms used can depend on the desired metal ion. In addition, in some embodiments, a single macrocycle can contain heteroatoms of different types.

In an illustrative embodiment, the macrocyclic component contains one metal ion; in some embodiments the macrocyclic complex comprises a single metal ion, although, polynucleate complexes, including polynucleate macrocyclic complexes, are also contemplated.

In another illustrative embodiment, the macrocyclic component is roughly based on porphyrins as described herein, including, but not limited to, a 16 member ring, 17 membered rings, 18 membered rings, 19 membered rings, or 20 membered rings, or combinations thereof, are all contemplated.

In another illustrative embodiment, the Q moiety forms 5 or 6 membered rings that are optionally substituted with 1 or 2 (in the case of 5 membered rings) or 1, 2, or 3 (in the case of 6 membered rings) with independently selected R groups as illustrated above. In some embodiments, the rings, bonds and substitutents are chosen to result in the compound being electronically conjugated, and in some cases have at a minimum have at least two oxidation states.

In some embodiments, the macrocyclic component of the invention is selected from the group consisting of porphyrins and cyclen derivatives.

A preferred group of macrocycles encompassed by the invention are porphyrins, including porphyrin derivatives. Porphyrin and certain porphyrin derivatives are encompassed by the formula:

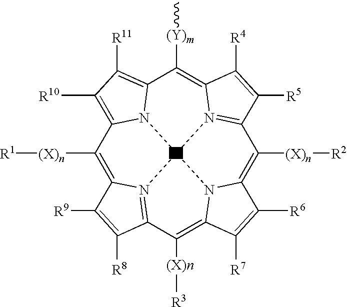

wherein X, Y, each of the R groups, M, n and m are defined above.

As is true for the compounds outlined herein, and as will be appreciated by those in the art, each unsaturated position, whether carbon or heteroatom, can include one or more substitution groups as defined herein, depending on the desired valency of the system. In general, useful embodiments have only one R substituent group per atom.

Particularly preferred substituents include, but are not limited to, 4-chlorophenyl, 3-acetamidophenyl, 2,4-dichloro-4-trifluoromethyl. Preferred substituents provide a redox potential range of less than about 2 volts. X is selected from the group consisting of a substrate, a reactive site that can covalently couple to a substrate (e.g. an alcohol, a thiol, etc.). It will be appreciated that in some embodiments, L-X may be an alcohol or a thiol. In certain instances L-X can be replaced with another substituent. In certain embodiments, L-X can be present or absent, and when present preferably is 4-hydroxyphenyl, 4-(2-(4-hydroxyphenyl)ethynyl)-phenyl, 4-hydroxymethyl)phenyl, 4-mercaptophenyl, 4-(2-(4-mercaptophenyl)ethynyl)phenyl, 4-(mercaptomethyl)phenyl, 4-hydroselenophenyl, 4-(2-(4-hydroselenophenyl)ethynyl)phenyl, 4-(hydroselenylmethyl)phenyl, 4-hydrotellurophenyl, 4-(2-(4-hydrotellurophenyl)ethynyl)phenyl, and 4-(hydrotelluromethyl)phenyl.

A class of molecules finding particular use in the invention as oxidizable or reducible species are ReAM based phosphines.

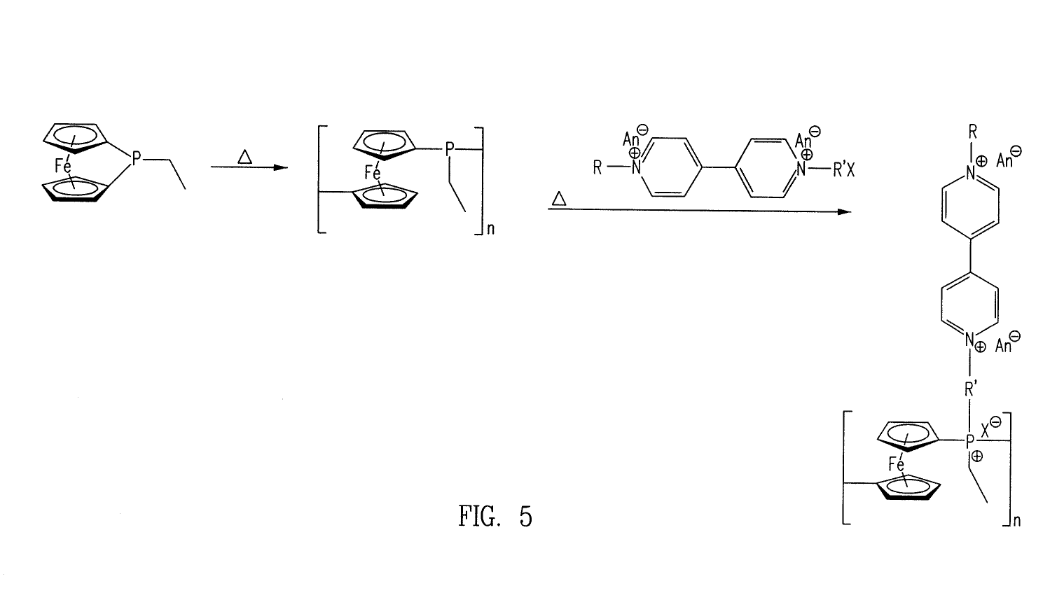

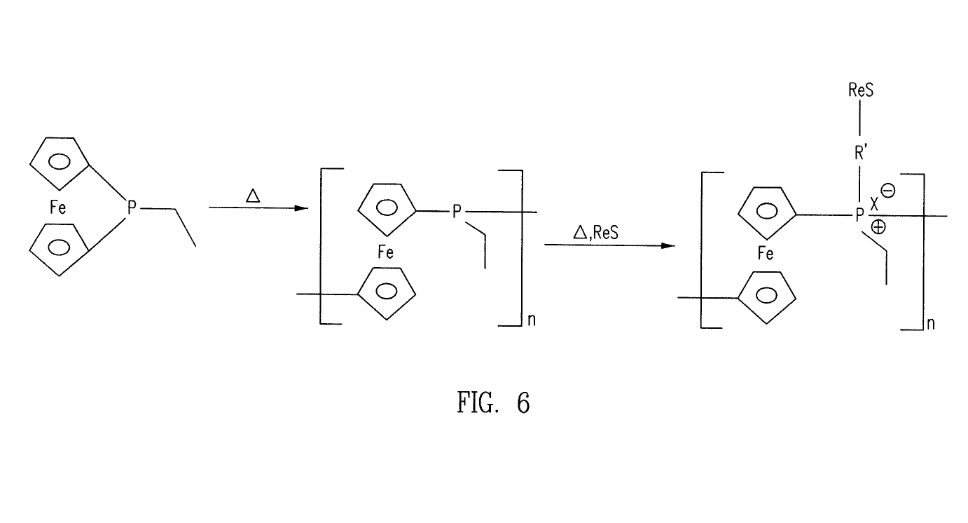

In this embodiment, a “phosphine” is a phosphate species with three covalent bonds to the phosphate heteroatom. In this embodiment, a phosphocyclophane (a cyclic phosphine) comprising a ReAM such as an oxidizable species (Ox), is reacted to form a polymer; e.g. the phosphocyclophane monomer is polymerized to form a reactive phosphine polymer, as generally depicted in FIGS. 5 and 6. This reactive phosphine polymer is then reacted with a reducible species (ReS), to form the compounds of the invention. Alternatively, the process can be reversed using the reducible species to form the initial phosphine, which is then reacted with an oxidizable species to form the polymer.

In one embodiment, the polymerization treatment is heat. Depending on the compositions selected, suitable heat treatment ranges include, but are not limited to, from 50 to 250° C., with from about 100° C. to about 150° C. being preferred in some embodiments. Alternative polymerization treatments include acid and base catalyzed polymerization, and photo-induced polymerization.

In one embodiment, the ReAM based phosphine is a metallocene derivative. FIG. 5 depicts an example using ferrocenyl phosphacyclophanes, which upon heat treatment, polymerize to form ferrocenyl phosphine polymers. These can then be reacted with any number of different ReS to form the compositions of the invention. FIG. 5 depicts a ferrocene phosphocylcophane using phosphine polymer example, while FIG. 6 is a more general depiction, utilizing a “generic” ReS.

In one embodiment, similar to the ReAM based phosphine chemistry, ether, sulfide, silane, amine or boranes may be used to form metallocene derivatives as well.

It should be recognized that other ReAMs, in addition to metallocenes, can be used as well in the phosphine, ether, sulfide, silane, amine or borane chemistries as well.

In addition, as will be appreciated by those in the art, there are a variety of methods that can utilize the phosphine chemistry (as well as the additional polymerization monomers, outlined below). In one embodiment, the phosphocyclophane monomers are deposited to optimal thickness, depending on the use of the compositions, as is described herein. The substrate is then heated to result in polymerization, and the resulting reactive phosphine is quaternized with a suitable reducible species (e.g. shown in FIG. 5 as a viologen). The thickness can vary depending on the type, morphology, etc. of the polymer generated, the degree of substitution desired for the addition of the counter-reaction moiety, the extent of incorporation of conducting species (ions), types of conducting species (ions) how much flexibility or mobility is desired, etc. to achieve the physicomechanical and/or electrical properties of the material.

In an alternate embodiment, successive depositions, interspersed with polymerization steps, may be used, with either the phosphine monomers or other monomers.

In the case where heteroatoms other than phosphate are used, e.g. if the heteroatom is boron, nitrogen, oxygen, sulfur or other 3A, 5A or 6A element, reaction with a suitable reagent will result in formation of a cationic heteroatom in the polymer chain, with concomitant liberation of an anion. Alternatively, if other group 4A elements comprise the heteroatom, e.g. Si, appropriate substitution of one or both of the C—, Si— or other group 4A atom-substitutents can result in the formation of a suitably conducting cation/anion pair.

In addition to phosphine polymers, the oxidizable species of the invention may also comprise polymers of the molecules described above; for example, porphyrin polymers (including polymers of porphyrin complexes), macrocycle complex polymers, molecules comprising two redox active subunits, etc. The polymers can be homopolymers or heteropolymers, and can include any number of different mixtures (admixtures) of monomeric molecules, wherein “monomer” can also include molecules comprising two or more subunits (e.g., a sandwich coordination compound, a porphyrin derivative substituted with one or more ferrocenes, etc.). ReAM polymers are described in U.S. Publication No. 20070123618, which is expressly incorporated by reference in its entirety.

The configuration of the polymers on the electrode can vary. In some embodiments, the polymers are linear in the Z dimension (the direction perpendicular to the substrate surface, as is depicted in FIG. 14A of U.S. Publication No. 20070123618), and can be optionally crosslinked (FIG. 14B). Branched polymers in the Z dimension are also contemplated, and can be optionally crosslinked as well. Linear polymers in the X-Y-dimension (FIG. 14C), or branched and/or crosslinked polymers are also included. In addition, mixtures of polymers can be used in any of these configurations.

In some embodiments, configurations (including selection of linkers) that control orientation and spacing of the oxidizable species, whether polymeric or monomeric, are preferred, as generally higher densities of the molecules can be achieved, as well as better electron transfer and electron transfer rates. Linker length can contribute to the rate and retention of charge

In general, the polymerization embodiments rely on the use of substitutents that will result in both attachment to the electrode surface as well as polymerization to additional redox active molecules. As described in U.S. Publication No. 20070123618, there are two general methods for the synthesis of these ReAMs: “in situ” polymerization on the surface, and prepolymerization followed by addition to the surface using one or more attachment moieties, described in detail in U.S. Publication No. 20070123618, expressly incorporated by reference in its entirety, and with particular respect herein as to the “one-step” and “two-step” polymerization/attachment steps.

5.2.2. The Reducible Functional Species

The reducible functional species of the self-contained charge storage unit is held in close proximity to an electrically, negatively-biased electrode, for example, the counter electrode. Preferably, the reducible functional species is chemically bound to an electrically, negatively-biased electrode; most preferably the reducible functional species is covalently bound to the electrically, negatively-biased electrode.

Generally, the reducible functional species that can be utilized as the reducible functional species of the self-contained charge storage unit can be any stable molecular species capable of being reduced within a suitable electrochemical window, generally described herein as a ReAM. Preferably, the reducible functional species includes those that are electronically conjugated and those that may not be.

As will be understood to those of skill in the art, the reducible functional species of the self-contained charge storage unit may be the same backbone as the oxidizable species (e.g., both a monomer or polymeric substituted porphyrins) and therefore any of the porphyrinic groups set forth in section 5.2.1. herein may qualify as the reducible species of the invention.

In a preferred embodiment, the reducible functional species of the self-contained charge storage unit is a polycyclic aromatic hydrocarbon nuclei or a porphyrinic species.

In one illustrative embodiment, the reducible functional species of the self-contained charge storage unit has the following formula:

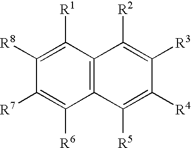

wherein each of R¹-R⁸ is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂; or adjacent R groups can form a 3-8 membered ring optionally substituted.

In one embodiment, at least one of R¹, R², R³, R⁴, R⁵, R⁶, R⁷, or R⁸ is —H. In another embodiment, at least one of R¹, R², R³, R⁴, R⁵, R⁶, R⁷, or R⁸ is —OH. In another embodiment, at least one of R¹, R², R³, R⁴, R⁵, R⁶, R⁷, or R⁸ is —N(C₁-C₁₀)alkyl(C₁-C₁₀)alkyl. In another embodiment, at least one of R¹, R², R³, R⁴, R⁵, R⁶, R⁷, or R⁸ is —(C₁-C₁₀)alkyl. In another embodiment, at least one of R¹, R², R³, R⁴, R⁵, R⁶, R⁷, or R⁸ is —O(C₁-C₁₀)alkyl. In another embodiment, at least one of R¹, R², R³, R⁴, R⁵, R⁶, R⁷, or R⁸ is —O—C(O)(C₁-C₁₀)alkyl. In another embodiment, at least one of R¹, R², R³, R⁴, R⁵, R⁶, R⁷ or R⁸ is —NH(CH₂)ₘ(C₁-C₁₀)alkyl. In another embodiment, at least one of R¹, R², R³, R⁴, R⁵, R⁶, R⁷, or R⁸ is —O—CF₃. In another embodiment, at least one of R¹, R², R³, R⁴, R⁵, R⁶, R⁷, or R⁸ is —O-benzyl. In another embodiment, at least one of R¹, R², R³, R⁴, R⁵, R⁶, R⁷, or R⁸ is —O—(CH₂)ₘCH((C₁-C₁₀)alkyl(C₁-C₁₀)alkyl). In another embodiment, at least one of R¹, R², R³, R⁴, R⁵, R⁶, R⁷, or R⁸ is —(C₂-C₁₀)alkenyl. In another embodiment, at least one of R¹, R², R³, R⁴, R⁵, R⁶, R⁷, or R⁸ is —(C₂-C₁₀)alkynyl. In another embodiment, at least one of R¹, R², R³, R⁴, R⁵, R⁶, R⁷, or R⁸ is —(C₃-C₁₀)cycloalkyl. In another embodiment, at least one of R¹, R², R³, R⁴, R⁵, R⁶, R⁷, or R⁸ is —(C₈-C₁₄)bicycloalkyl. In another embodiment, at least one of R¹, R², R³, R⁴, R⁵, R⁶R⁷, or R⁸ is —(C₅-C₁₀)cycloalkenyl. In another embodiment, at least one of R¹, R², R³, R⁴, R⁵, R⁶, R⁷, or R⁸ is —O—(C₂-C₁₀)alkenyl. In another embodiment, at least one of R¹, R², R³, R⁴, R⁵, R⁶, R⁷, or R⁸ is —O—(C₂-C₁₀)alkynyl. In another embodiment, at least one of R¹, R², R³, R⁴, R⁵, R⁶, R⁷, or R⁸ is —O—(C₃-C₁₀)cycloalkyl. In another embodiment, at least one of R¹, R², R³, R⁴, R⁵, R⁶, R⁷, or R⁸ is —O—(C₈-C₁₄)bicycloalkyl. In another embodiment, at least one of R¹, R², R³, R⁴, R⁵, R⁶, R⁷, or R⁸ is —O—(C₅-C₁₀)cycloalkenyl. In another embodiment, at least one of R¹, R², R³, R⁴, R⁵, R⁶, R⁷, or R⁸ is —O-heteroaryl. In another embodiment, at least one of R¹, R², R³, R⁴, R⁵, R⁶, R⁷ or R⁸ is -heteroaryl. In another embodiment, at least one of R¹, R²R³, R⁴, R⁵, R⁶, R⁷, or R⁸ is -aryl. In another embodiment, at least one of R¹, R², R³, R⁴, R⁵, R⁶, R⁷, or R⁸ is —(C₃-C₁₀)heterocyclealkyl. In another embodiment, at least one of R¹, R², R³, R⁴, R⁵, R⁶, R⁷, or R⁸ is —O—(CH₂)ₙ-aryl. In another embodiment, at least one of R¹, R², R³, R⁴, R⁵, R⁶, R⁷, or R⁸ is —O—(C₃-C₁₀)heterocycloalkyl. In another embodiment, at least one of R¹, R², R³, R⁴, R⁵, R⁶, R⁷, or R⁸ is —NHC(O)(C₁-C₁₀)alkyl. In another embodiment, at least one of R¹, R², R³, R⁴, R⁵, R⁶, R⁷, or R⁸ is —NHC(O)NH(C₁-C₁₀)alkyl. In another embodiment, at least one of R¹, R², R³, R⁴, R⁵, R⁶, R⁷, or R⁸ is —NH(aryl). In another embodiment, at least one of R¹, R², R³, R⁴, R⁵, R⁶, R⁷, or R⁸ is —N═C(aryl). In another embodiment, at least one of R¹, R², R³, R⁴, R⁵, R⁶, R⁷, or R⁸ is —SO₂NH₂. In another embodiment, at least R¹, R² and the naphthyl group form a six membered ring. In another embodiment, at least R⁵, R⁶ and the naphthyl group form a six membered ring. In another embodiment, at least R³, R⁴ and the naphthyl group form a six membered ring. In another embodiment, at least R⁷, R⁸ and the naphthyl group form a six membered ring.

In a preferred embodiment, the reducible functional species of the self-contained charge storage unit has the following formula:

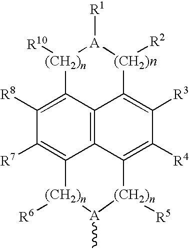

wherein

A is independently N, O, S, Se, Te, CH;

R¹-R¹⁰ are each independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂; optionally R², R⁵, R⁶, and R¹⁰ can be a ═O group;

n is 0-5; and

[symbol] is an attachment, preferably covalent to the oxidizable species or the ionic conducting electrolyte.

In one embodiment, at least one A is —N. In another embodiment, at least one A is O.

In another embodiment, at least one A is S. In another embodiment, at least one A is Se. In another embodiment, at least one A is Te. In another embodiment, at least one A is CH or CH₂.

In one embodiment, at least one n is 0. In another embodiment, at least one n is 1. In another embodiment, at least one n is 2. In another embodiment, at least one n is 3. In another embodiment, at least one n is 4. In another embodiment, at least one n is 5.

In one embodiment, at least one of R¹, R², R³, R⁴, R⁵, R⁶, R⁷, R⁸, R⁹, or R¹⁰ is —H. In another embodiment, at least one of R¹, R², R³, R⁴, R⁵, R⁶, R⁷, R⁸, R⁹, or R¹⁰ is —OH. In another embodiment, at least one of R¹, R², R³, R⁴, R⁵, R⁶, R⁷, R⁸, R⁹, or R¹⁰ is —N(C₁-C₁₀)alkyl(C₁-C₁₀)alkyl. In another embodiment, at least one of R¹, R², R³, R⁴, R⁵, R⁶, R⁷, R⁸, R⁹, or R¹⁰ is —(C₁-C₁₀)alkyl. In another embodiment, at least one of R¹, R², R³, R⁴, R⁵, R⁶, R⁷, R⁸, R⁹, or R¹⁰ is —O(C₁-C₁₀)alkyl. In another embodiment, at least one of R¹, R², R³, R⁴, R⁵, R⁶, R⁷, R⁸, R⁹, or R¹⁰ is —O—C(O)(C₁-C₁₀)alkyl. In another embodiment, at least one of R¹, R², R³, R⁴, R⁵, R⁶, R⁷, R⁸, R⁹, or R¹⁰ is —NH(CH₂)ₘ(C₁-C₁₀)alkyl. In another embodiment, at least one of R¹, R², R³, R⁴, R⁵, R⁶, R⁷, R⁸, R⁹, or R¹⁰ is —O—CF₃. In another embodiment, at least one of R¹, R², R³, R⁴, R⁵, R⁶, R⁷, R⁸, R⁹, or R¹⁰ is —O-benzyl. In another embodiment, at least one of R¹, R², R³, R⁴, R⁵, R⁶, R⁷, R⁸, R⁹, or R¹⁰ is —O—(CH₂)ₘCH((C₁-C₁₀)alkyl(C₁-C₁₀)alkyl). In another embodiment, at least one of R¹, R², R³, R⁴, R⁵, R⁶, R⁷, R⁸, R⁹, or R¹⁰ is —(C₂-C₁₀)alkenyl. In another embodiment, at least one of R¹, R², R³, R⁴, R⁵, R⁶, R⁷, R⁸, R⁹, or R¹⁰ is —(C₂-C₁₀)alkynyl. In another embodiment, at least one of R¹, R², R³, R⁴, R⁵, R⁶, R⁷, R⁸, R⁹, or R¹⁰ is —(C₃-C₁₀)cycloalkyl. In another embodiment, at least one of R¹, R², R³, R⁴, R⁵, R⁶, R⁷, R⁸, R⁹, or R¹⁰ is —(C₈-C₁₄)bicycloalkyl. In another embodiment, at least one of R¹, R², R³, R⁴, R⁵, R⁶, R⁷, R⁸, R⁹, or R¹⁰ is —(C₅-C₁₀)cycloalkenyl. In another embodiment, at least one of R¹, R², R³, R⁴, R⁵, R⁶, R⁷, R⁸, R⁹, or R¹⁰ is —O—(C₂-C₁₀)alkenyl. In another embodiment, at least one of R¹, R², R³, R⁴, R⁵, R⁶, R⁷, R⁸, R⁹, or R¹⁰ is —O—(C₂-C₁₀)alkynyl. In another embodiment, at least one of R¹, R², R³, R⁴, R⁵, R⁶, R⁷, R⁸, R⁹, or R¹⁰ is —O—(C₃-C₁₀)cycloalkyl. In another embodiment, at least one of R¹, R², R³, R⁴, R⁵, R⁶, R⁷, R⁸, R⁹, or R¹⁰ is —O—(C₈-C₁₄)bicycloalkyl. In another embodiment, at least one of R¹, R², R³, R⁴, R⁵, R⁶, R⁷, R⁸, R⁹, or R¹⁰ is —O—(C₅-C₁₀)cycloalkenyl. In another embodiment, at least one of R¹, R², R³, R⁴, R⁵, R⁶, R⁷, R⁸, R⁹, or R¹⁰ is —O-heteroaryl. In another embodiment, at least one of R¹, R², R³, R⁴, R⁵, R⁶, R⁷, R⁸, R⁹, or R¹⁰ is -heteroaryl. In another embodiment, at least one of R¹, R², R³, R⁴, R⁵, R⁶, R⁷, R⁸R⁹, or R¹⁰ is -aryl. In another embodiment, at least one of R¹, R², R³, R⁴, R⁵, R⁶, R⁷, R⁸, R⁹, or R¹⁰ is —(C₃-C₁₀)heterocyclealkyl. In another embodiment, at least one of R¹, R², R³, R⁴, R⁵, R⁶, R⁷, R⁸, R⁹, or R¹⁰ is —O—(CH₂)ₙ-aryl. In another embodiment, at least one of R¹, R², R³, R⁴, R⁵, R⁶, R⁷, R⁸, R⁹, or R¹⁰ is —O—(C₃-C₁₀)heterocycloalkyl. In another embodiment, at least one of R¹, R², R³, R⁴, R⁵, R⁶, R⁷, R⁸, R⁹, or R¹⁰ is —NHC(O)(C₁-C₁₀)alkyl. In another embodiment, at least one of R¹, R², R³, R⁴, R⁵, R⁶, R⁷, R⁸, R⁹, or R¹⁰ is —NHC(O)NH(C₁-C₁₀)alkyl. In another embodiment, at least one of R¹, R², R³, R⁴, R⁵, R⁶, R⁷, R⁸, R⁹, or R¹⁰ is —NH(aryl). In another embodiment, at least one of R¹, R², R³, R⁴, R⁵, R⁶, R⁷, R⁹, or R¹⁰ is —N═C(aryl). In another embodiment, at least one of R¹, R², R³, R⁴, R⁵, R⁶, R⁷, R⁸, R⁹, or R¹⁰ is —C≡N. In another embodiment, at least one of R¹, R², R³, R⁴, R⁵, R⁶, R⁷, R⁸, R⁹, or R¹⁰ is —SO₂NH₂.

In another preferred embodiment, the reducible functional species of the self-contained charge storage unit has the following formula:

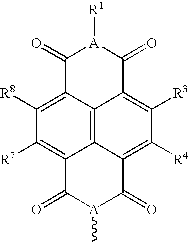

wherein each A and the R groups are defined above.

In another preferred embodiment, the reducible functional species of the self-contained charge storage unit has the following formula:

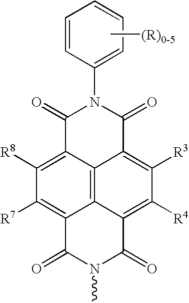

wherein the R groups are defined above.

5.2.4. The Ionic Conducting Electrolyte

The ionic conducting electrolyte of the self-contained charge storage unit is preferably devoid of any appreciable electrical conductivity, preferably physically separates the oxidizable species from the reducible species and is preferably in close enough contact with both the oxidizable species and the reducible species that charge development of both species may occur, preferably simultaneously.

Preferably, the ionic conducting electrolyte of the self-contained charge storage unit is a solid, a liquid or a combination of solid and liquid possessing the required ionic conductivity that is inserted into or otherwise produced in situ in the physical space separating the oxidizable species and the reducible species.

The ionic conducting electrolyte of the self-contained charge storage unit can be both covalently bound cationic or anionic species with requisite counter ions for charge neutrality.

In one illustrative embodiment, the ionic conducting electrolyte has the formula:

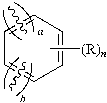

wherein each R is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —(P((C₁-C₁₀)alkyl)₃)+, —SO₃—, —SO₂NH₂;

n is 0-4;

( [symbol] )ₐ is an attachment to either the oxidizable species or the reducible species; and

( [symbol] )_{b} is an optional attachment to either the oxidizable species or the reducible species.

In one embodiment, at least one R is —H. In another embodiment, at least one R is —OH. In another embodiment, at least one R is —N(C₁-C₁₀)alkyl(C₁-C₁₀)alkyl. In another embodiment, at least one R is —(C₁-C₁₀)alkyl. In another embodiment, at least one R is —O(C₁-C₁₀)alkyl. In another embodiment, at least one R is —O—C(O)(C₁-C₁₀)alkyl. In another embodiment, at least one R is —NH(CH₂)ₘ(C₁-C₁₀)alkyl. In another embodiment, at least one R is —O—CF₃. In another embodiment, at least one R is —O-benzyl. In another embodiment, at least one R is —O—(CH₂)ₘCH((C₁-C₁₀)alkyl(C₁-C₁₀)alkyl). In another embodiment, at least one R is —(C₂-C₁₀)alkenyl. In another embodiment, at least one R is —(C₂-C₁₀)alkynyl. In another embodiment, at least one R is —(C₃-C₁₀)cycloalkyl. In another embodiment, at least one R is —(C₈-C₁₄)bicycloalkyl. In another embodiment, at least one R is —(C₅-C₁₀)cycloalkenyl. In another embodiment, at least one R is —O—(C₂-C₁₀)alkenyl. In another embodiment, at least one R is —O—(C₂-C₁₀)alkynyl. In another embodiment, at least one R is —O—(C₃-C₁₀)cycloalkyl. In another embodiment, at least one R is —O—(C₈-C₁₄)bicycloalkyl. In another embodiment, at least one R is —O—(C₅-C₁₀)cycloalkenyl. In another embodiment, at least one R is —O-heteroaryl. In another embodiment, at least one R is -heteroaryl. In another embodiment, at least one R is -aryl. In another embodiment, at least one R is —(C₃-C₁₀)heterocyclealkyl. In another embodiment, at least one R is —O—(CH₂)ₙ-aryl. In another embodiment, at least one R is —O—(C₃-C₁₀)heterocycloalkyl. In another embodiment, at least one R is —NHC(O)(C₁-C₁₀)alkyl. In another embodiment, at least one R is —NHC(O)NH(C₁-C₁₀)alkyl. In another embodiment, at least one R is —NH(aryl). In another embodiment, at least one R is —N═C(aryl). In another embodiment, at least one R is —(P((C₁-C₁₀)alkyl)₃)⁺. In another embodiment, at least one R is —SO₃⁻. In another embodiment, at least one R is —SO₂NH₂.

In one embodiment, n is 0. In another embodiment, n is 1. In one embodiment, n is 2. In one embodiment, n is 3. In one embodiment, n is 4.

In a preferred embodiment, the ionic conducting electrolyte of the self-contained charge storage unit has the following formula:

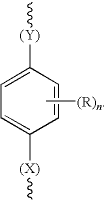

wherein X and Y are linkages between the oxidizable species and the reducible species, wherein the linkages vary in type, length and functionality.

In another preferred embodiment, the ionic conducting electrolyte of the self-contained charge storage unit has the following formula:

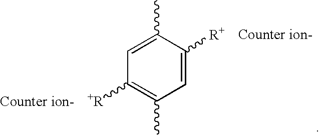

wherein R is defined above. Preferably, in one embodiment each R is a —(P((C₁-C₁₀)alkyl)₃)⁺ group.

In a particular embodiment, the ionic conducting electrolyte of the self-contained charge storage unit has the following formula:

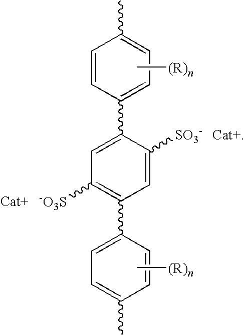

wherein R and n are defined above and Cat⁺ is any suitable cation and ( [symbol] ) is an optional attachment to either the oxidizable species or the reducible species, which vary in type, length and functionality.

In a more particular embodiment, the ionic conducting electrolyte of the self-contained charge storage unit has the following formula:

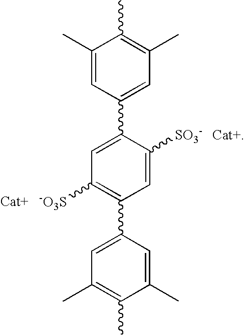

In another particular embodiment, the ionic conducting electrolyte of the self-contained charge storage unit has the following formula:

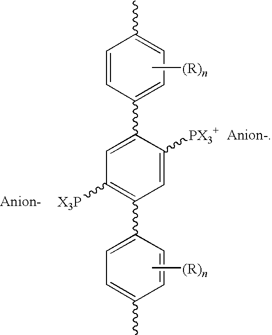

wherein R and n are defined above and X is an alkyl group and Anion- is any suitable counter anion, preferably imidazole and ( [symbol] ) is an optional linkage, which vary in type, length and functionality.

In another particular embodiment, the ionic conducting electrolyte of the self-contained charge storage unit has the following formula:

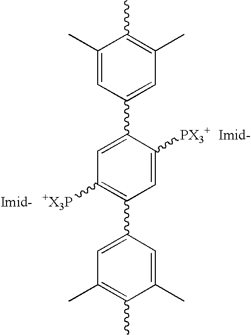

wherein X is an alkyl group.

5.2.4. Linkers of the Invention

Linkers are used in a variety of configurations in the present invention, including to link attachment moieties to the redox active molecules of the invention, for linking together redox a active subunits of redox active molecules, and in polymerization of redox active molecules. Linkers are used in order to achieve fast writing and/or erasing at low voltages and a small cell size, the scaling of the linkers for use in the present invention can be optimized. Optimum linker size can be calculated theoretically (see U.S. Ser. No. 60/473,782, hereby expressly incorporated). Alternatively linkers (and in fact, the suitability of molecules as well) can be evaluated empirically simply by coupling the molecules of the invention to the surface as described herein and in the cited references, and performing voltammetry to evaluate the electrochemical properties of the attached polymer.

### 5.3. Illustrative Self-Contained Charge Storage Molecules of the Invention

As set forth above, molecules encompassed by the invention generally have one or more reducible species are attached to one or more ion conducting moieties and the one or more ion conducting moieties are then attached to one or more oxidizable species components in the following manner:

(ReS)ₗ-(IC)ₘ-(Ox)ₙ.

In an illustrative embodiment of the invention, a porphyrin moiety can act as the oxidizable and reducible species as illustrated by the following formula:

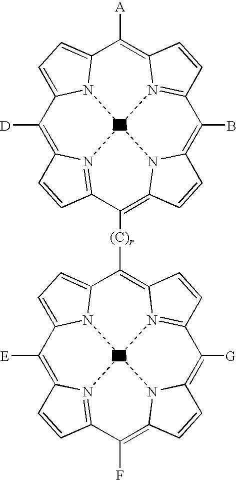

wherein A, B, D, E, F, and G are suitable substituents and C is an ionic conducting electrolyte. One of skill in the art will recognize that the substitution on the above molecule can be varied to effect, for example, read/write speeds, packing density, and processability. For example, the substitution of the above molecule can be varied as follows:

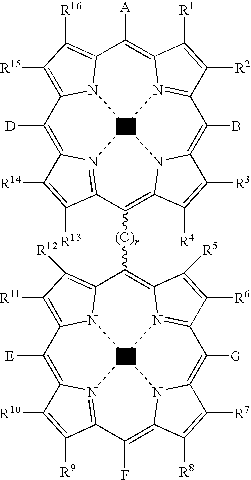

wherein A, B, D, E, F, G; the R groups, and the metal M can be varied to effect the desired properties of the molecule. The [symbol] group type, length, and functionality can be varied to effect changes in the read/write speed or other properties of the molecule or device.

In a particularly preferred embodiment, the self-contained charge storage molecule of the invention has the formula:

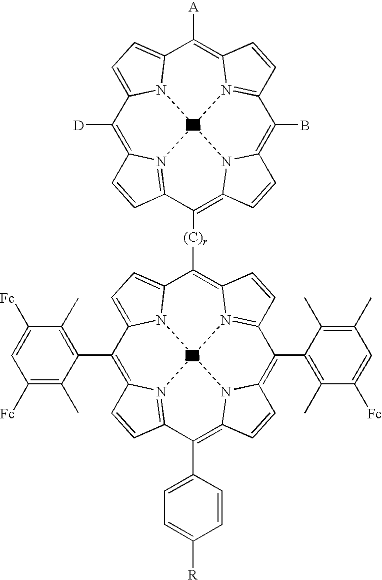

wherein Fe is a ferrocene substituent. Ferrocene substituents provide a convenient series of one-bit molecules having different and distinguishable oxidation states. Thus the molecules of these formulae have various oxidation potentials (e.g., +0.55 V, +0.48V, +0.39 V, +0.17 V, −0.05 V, and −0.18 V) to provide a convenient series of molecules for incorporation into a storage medium of this invention. It will be appreciated that the oxidation potentials of the members of the series can be routinely altered by changing the metal (M) or the substituents.

In another illustrative embodiment of the invention, the oxidizable species is a porphyrin and reducible species is any suitably reducible species, preferably a napthyl moiety as illustrated by the following formula:

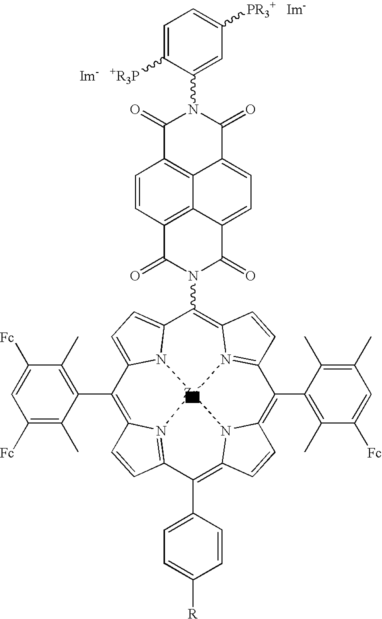

wherein the ion conducting moiety is illustrated as the substituted benzene, the phenanthroline-tetraone portion is the reducible species, and substituted porphyrin is the oxidizable species.

In addition, the above identified structure further illustrates that the order of attachment of the oxidizable species, the reducible species and the ionic conducting electrolyte is not essential.

Illustrative embodiments of the self-contained charge storage molecules of the invention are illustrated below:

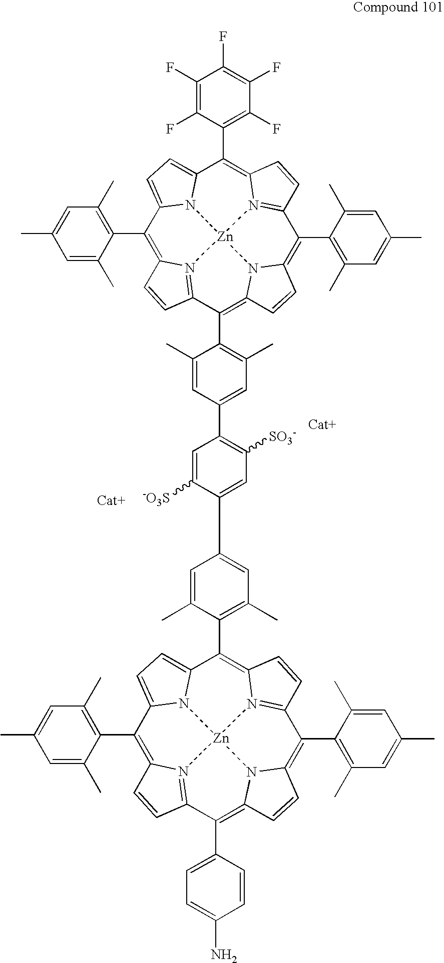

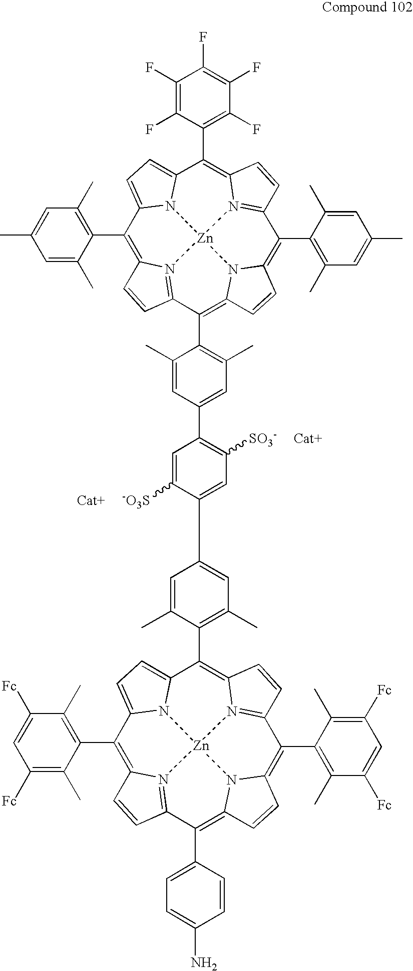

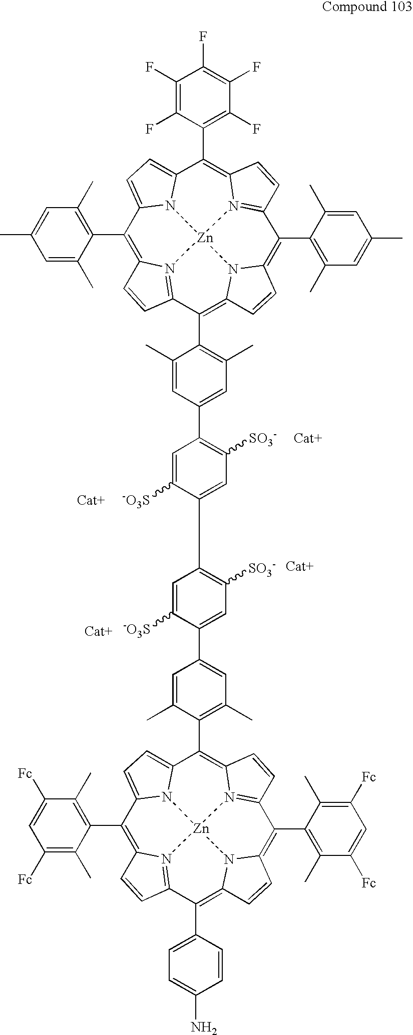

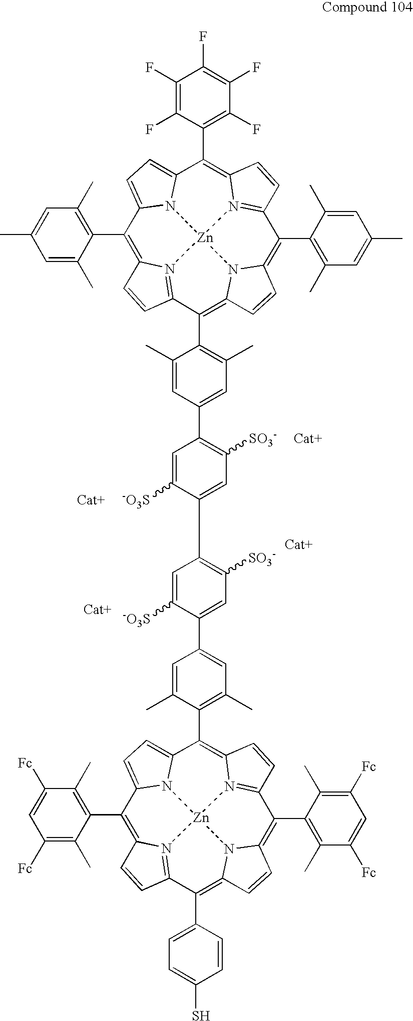

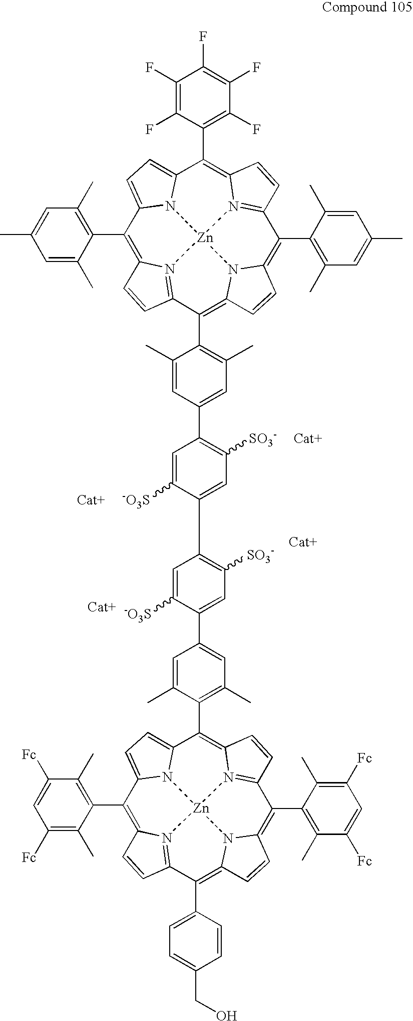

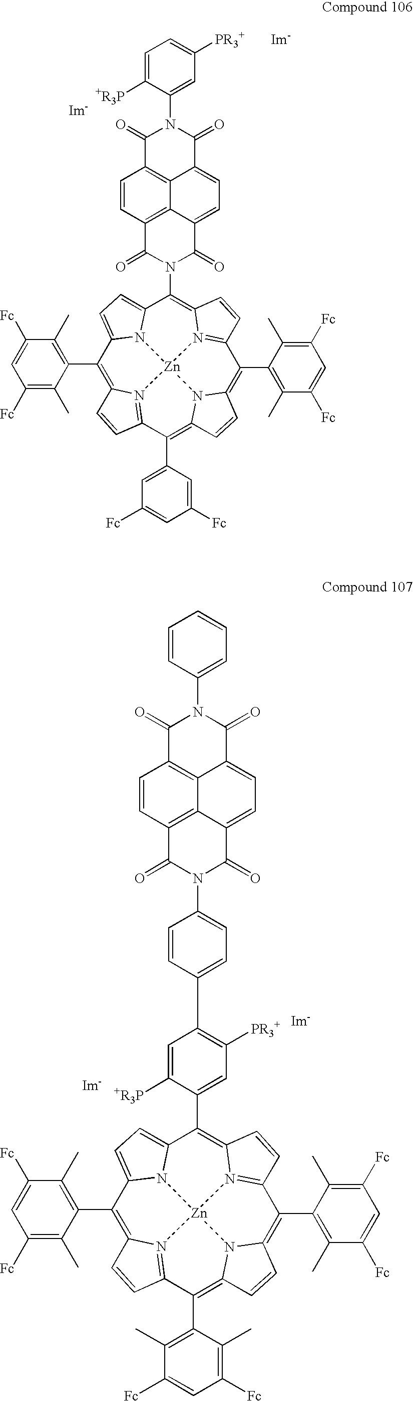

### 5.4. Uses of the Self-Contained Charge Storage Molecules of the Invention

Control over the hole-storage and hole-hopping properties of the molecules used in the memory devices of the present invention allows fine control over the architecture of the memory device.

Such control is exercised through synthetic design. The hole-storage properties depend on the oxidation potential of the redox-active units or subunits that are themselves or are that are used to assemble the storage media used in the devices of this invention. The hole-storage properties and redox potential can be tuned with precision by choice of base molecule(s), associated metals and peripheral substituents (Yang et al. (1999) J. Porphyrins Phthalocyanines, 3: 117-147), the disclosure of which is herein incorporated by this reference.

For example, in the case of porphyrins, Mg porphyrins are more easily oxidized than Zn porphyrins, and electron withdrawing or electron releasing aryl groups can modulate the oxidation properties in predictable ways. Hole-hopping occurs among isoenergetic porphyrins in a nanostructure and is mediated via the covalent linker joining the porphyrins (Seth et al. (1994) J. Am. Chem. Soc., 116: 10578-10592, Seth et al (1996) J. Am. Chem. Soc., 118: 11194-11207, Strachan et al. (1997) J. Am. Chem. Soc., 119: 11191-11201; Li et al. (1997) J. Mater. Chem., 7: 1245-1262, Strachan et al. (1998) Inorg. Chem., 37: 1191-1201, Yang et al. (1999) J. Am. Chem. Soc., 121: 4008-4018), the disclosures of which are herein specifically incorporated by this reference in their entirety.

The design of compounds with predicted redox potentials is well known to those of ordinary skill in the art. In general, the oxidation potentials of redox-active units or subunits are well known to those of skill in the art and can be looked up (see, e.g., Handbook of Electrochemistry of the Elements). Moreover, in general, the effects of various substituents on the redox potentials of a molecule are generally additive. Thus, a theoretical oxidation potential can be readily predicted for any potential data storage molecule. The actual oxidation potential, particularly the oxidation potential of the information storage molecule(s) or the information storage medium can be measured according to standard methods. Typically the oxidation potential is predicted by comparison of the experimentally determined oxidation potential of a base molecule and that of a base molecule bearing one substituent in order to determine the shift in potential due to that particular substituent. The sum of such substituent-dependent potential shifts for the respective substituents then gives the predicted oxidation potential.

The suitability of particular molecules for use in the methods of this invention can readily be determined. The molecule(s) of interest are simply polymerized and coupled to a surface (e.g., a hydrogen passivated surface) according to the methods of this invention. Then sinusoidal voltammetry can be performed (e.g., as described herein or in U.S. Pat. Nos. 6,272,038; 6,212,093; and 6,208,553, PCT Publication WO 01/03126, or by (Roth et al. (2000) Vac. Sci. Technol. B 18:2359-2364; Roth et al. (2003) J. Am. Chem. Soc. 125:505-517) to evaluate 1) whether or not the molecule(s) coupled to the surface, 2) the degree of coverage (coupling); 3) whether or not the molecule(s) are degraded during the coupling procedure, and 4) the stability of the molecule(s) to multiple read/write operations.

The present invention contemplates a number of device and system architectures that enable the use of molecular memory elements (sometimes referred to herein and in related applications as “storage molecules” or “memory molecules”) and molecular charge storage devices to be integrated with and benefit from the technology developed for conventional microelectronics. At one level the present invention involves molecular memory elements that integrate storage molecules with switching logic that enables reading and writing to the storage molecules. At another extreme, the represent invention contemplates highly integrated circuits having logic devices, including data processing circuitry, integrated monolithically with molecular memory arrays to provide new and useful functions that leverage one or more the unique features of molecular memory such as lower power usage, superior data persistence, higher information density, and the like.

The design of compounds with predicted redox potentials is well known to those of ordinary skill in the art. In general, the oxidation potentials of redox-active units or subunits are well known to those of skill in the art and can be looked up (see, e.g., Handbook of Electrochemistry of the Elements). Moreover, in general, the effects of various substituents on the redox potentials of a molecule are generally additive. Thus, a theoretical oxidation potential can be readily predicted for any potential data storage molecule. The actual oxidation potential, particularly the oxidation potential of the information storage molecule(s) or the information storage medium can be measured according to standard methods. Typically the oxidation potential is predicted by comparison of the experimentally determined oxidation potential of a base molecule and that of a base molecule bearing one substituent in order to determine the shift in potential due to that particular substituent. The sum of such substituent-dependent potential shifts for the respective substituents then gives the predicted oxidation potential.

The suitability of particular redox-active molecules for use in the methods of this invention can readily be determined. The molecule(s) of interest are simply polymerized and coupled to a surface (e.g., a hydrogen passivated surface) according to the methods of this invention. Then sinusoidal voltammetry can be performed (e.g., as described herein or in U.S. Pat. Nos. 6,272,038; 6,212,093; and 6,208,553, PCT Publication WO 01/03126, or by (Roth et al. (2000) Vac. Sci. Technol. B 18:2359-2364; Roth et al. (2003) J. Am. Chem. Soc. 125:505-517) to evaluate 1) whether or not the molecule(s) coupled to the surface, 2) the degree of coverage (coupling); 3) whether or not the molecule(s) are degraded during the coupling procedure, and 4) the stability of the molecule(s) to multiple read/write operations.

5.4.1. Attachment Moieties

Attachment moieties are used to attach the molecules of the invention to one or more electrodes. Molecules bearing an attachment group include molecules wherein the attachment group is an intrinsic component of the molecule, molecules derivatized to add an attachment group, and molecules derivatized so they bear a linker comprising an attachment group.

The nature of the attachment moiety depends on the composition of the electrode substrate. In general, attachment moieties, together with the linkers, if present, allow the electronic coupling of the storage molecule to the electrode. Electronic coupling in this context refers to an association between the self-contained charge storage molecule and the electrode such that electrons move from the storage medium/molecule to the electrode or from the electrode to the storage medium/molecule and thereby alter the oxidation state of the storage medium/molecule. Electrical coupling can include direct covalent linkage between the storage medium/molecule and the electrode, indirect covalent coupling (e.g., via a linker), direct or indirect ionic bonding between the storage medium/molecule and the electrode, or other bonding (e.g., hydrophobic bonding). In addition, no actual bonding may be required and the storage medium/molecule may simply be contacted with the electrode surface. There also need not necessarily be any contact between the electrode and the storage medium/molecule where the electrode is sufficiently close to the storage medium/molecule to permit electron tunneling between the medium/molecule and the electrode.

Generally, suitable attachment moieties include, but are not limited to, carboxylic acids, alcohols, thiols (including S-acetylthiols) selenol, tellurol, phosphonic acids, phosphonothioate, amines, nitrile, aryl and alkyl groups, including substituted aryl and alkyl groups such as iodoaryls and bromomethyls. U.S. Publication No. 20070123618, incorporated by reference herein for this purpose, provides an extensive list of suitable attachment moieties and linkers (both independently and as “L-Z” groups); see paragraphs 107 to 113). It should be noted that the attachment moieties can result in a molecule (or linker attached to a molecule) being attached via a single group (e.g., “monopodal” attachment) or multiple groups (“polypodal” attachment). In some embodiments, polypodal attachment, such as tripodal attachment, results in a more fixed orientation of the molecules of the invention (including polymers) which can lead to higher density and cleaner signals. Polypodal (including tripodal) attachment moieties utilizing thiol, carboxylic acid, alcohol or phosphonic acids are particularly attractive. As outlined in the referenced application, some embodiments utilize attachment moieties based on triphenylmethane or tetraphenylmethane units, wherein 2 or 3 of the phenyl units are substituted with a suitable functional group for attachment (e.g., thiols such as Z-acetylthiol, or dihydroxylphosphoryl groups).

5.4.2. Electrodes

The self-contained charge storage molecules of the invention are electrically coupled to electrodes. The term “electrode” refers to any medium capable of transporting charge (e.g., electrons) to and/or from a storage molecule. Preferred electrodes are metals and conductive organic molecules, including, but not limited to, Group III elements (including doped and oxidized Group III elements), Group IV elements (including doped and oxidized Group IV elements), Group V elements (including doped and oxidized Group V elements) and transition metals (including transition metal oxides and transition metal nitrides). The electrodes can be manufactured to virtually and 2-dimensional or 3-dimensional shape (e.g., discrete lines, pads, planes, spheres, cylinders).

5.4.3 Fabrication and Characterization of the Storage Device

The memory devices of this invention can be fabricated using standard methods well known to those of skill in the art. In a preferred embodiment, the electrode layer(s) are applied to a suitable substrate (e.g., silica, glass, plastic, ceramic, etc.) according to standard well known methods (see, e.g., Rai-Choudhury (1997) The Handbook of Microlithography, Micromachining, and Microfabrication, SPIE Optical Engineering Press; Bard & Faulkner (1997) Fundamentals of Microfabrication). A variety of techniques are described below and also in U.S. Pat. Nos. 6,212,093; 6,728,129; 6,451,942; 6,777,516; 6,381,169; 6,208,553; 6,657,884; 6,272,038; 6,484,394; and U.S. Ser. Nos. 10/040,059; 10/682,868; 10/445,977; 10/834,630; 10/135,220; 10/723,315; 10/456,321; 10/376,865; and U.S. Publication No. 20070123618, all of which are expressly incorporated by reference, in particular for the fabrication techniques outlined therein.

5.4.4. Architectures

As outlined herein, the charge storage molecules are assembled into storage media, generally as components of electrochemical cells. The term “storage medium” refers to a composition comprising two or more storage molecules. The storage medium can contain only one species of storage molecule or it can contain two or more different species of storage molecule. In preferred embodiments, the term “storage medium” refers to a collection of storage molecules. Preferred storage media comprise a multiplicity (at least 2) of different and distinguishable (preferably non-neutral) oxidation states. The multiplicity of different and distinguishable oxidation states can be produced by the combination of different species of storage molecules, each species contributing to the multiplicity of different oxidation states and each species having a single non-neutral oxidation state. Alternatively or in addition, the storage medium can comprise one or more species of storage molecule having a multiplicity of non-neutral oxidation states. The storage medium can contain predominantly one species of storage molecule or it can contain a number of different storage molecules. The storage media can also include molecules other than storage molecules (e.g., to provide chemical stability, suitable mechanical properties, to prevent charge leakage, etc.).

There are a wide variety of device and systems architectures that benefit from the use of molecular memory. In one embodiment a molecular charge storage device is provided, comprising: a working electrode and a counter electrode configured to afford electrical capacitance; and one or more self-contained charge storage molecules positioned between said working and counter electrodes, said self-contained charge storage molecules comprised of one or more reducible functional species (ReS); one or more oxidizable functional species (Ox); and one or more ion conducting moiety (IC).

Memory devices are operated by receiving an N-bit row address into row address decoder and an M-bit column address into column address decoder. The row address decoder generates a signal on one word line. Word lines may include word line driver circuitry that drives a high current signal onto word lines. Because word lines tend to be long, thin conductors that stretch across much of the chip surface, it requires significant current and large power switches to drive a word lines signal. As a result, line driver circuits are often provided with power supply in addition to power supply circuits (not shown) that provide operating power for the other logic. Word line drivers, therefore, tend to involve large components and the high speed switching of large currents tends to create noise, stress the limits of power supplies and power regulators, and stress isolation structures.

In a conventional memory array there are more columns (bit lines) than rows (word lines) because during refresh operations, each word line is activated to refresh all of storage elements coupled to that word line. Accordingly, the fewer the number of rows, the less time it takes to refresh all of the rows. One feature of the present invention is that the molecular memory elements can be configured to exhibit significantly longer data retention than typical capacitors, in the order of tens, hundreds, thousands or effectively, unlimited seconds. Hence, the refresh cycle can be performed orders of magnitude less frequently or omitted altogether. Accordingly, refresh considerations that actually affect the physical layout of a memory array can be relaxed and arrays of various geometry can be implemented. For example, memory array can readily be manufactured with a larger number of word lines, which will make each word line shorter. As a result, word line driver circuits can be made smaller or eliminated because less current is required to drive each word line at a high speed. Alternatively or in addition, shorter word lines can be driven faster to improve read/write access times. As yet another alternative, each row of memory locations can be provided with multiple word lines to provide a mechanism for storing multiple states of information in each memory location

Sense amplifiers are coupled to each bit line and operate to detect signals on bit lines 109 that indicate the state of a memory element coupled to that bit line, and amplify that state to an appropriate logic level signal. In one embodiment, sense amplifiers may be implemented with substantially conventional designs as such conventional designs will operate to detect and amplify signals from a molecular memory element. Alternatively, unlike conventional capacitors, some molecular storage elements provide very distinct signals indicating their state. These distinct signals may reduce the need for conventional sense amplifier logic as the state signal from a molecular storage device can be more readily and reliably latched into buffers of read/write logic than can signals stored in conventional capacitors. That is, the present invention can provide devices which are sufficiently large as to obviate the need for a sense amplifier.

Read/write logic includes circuitry for placing the memory device in a read or write state. In a read state, data from molecular array is placed on bit lines (with or without the operation of sense amplifiers), and captured by buffers/latches in read/write logic. Column address decoder will select which bit lines are active in a particular read operation. In a write operation, read/write logic drives data signals onto the selected bit lines such that when a word line is activated, that data overwrites any data already stored in the addressed memory element(s).

A refresh operation is substantially similar to a read operation; however, the word lines are driven by refresh circuitry (not shown) rather than by externally applied addresses. In a refresh operation, sense amplifiers, if used, drive the bit lines to signal levels indicating the current state of the memory elements and that value is automatically written back to the memory elements. Unlike a read operation, the state of bit lines is not coupled to read/write logic during a refresh. This operation is only required if the charge retention time of the molecules used is less than the operational life of the device used, for example, on the order of 10 years for Flash memory.

In an exemplary embedded system that comprises a central processing unit and molecular memory, a memory bus couples a CPU and molecular memory device to exchange address, data, and control signals. Optionally, embedded system may also contain conventional memory coupled to memory bus. Conventional memory may include random access memory (e.g., DRAM, SRAM, SDRAM and the like), or read only memory (e.g., ROM, EPROM, EEPROM and the like). These other types of memory may be useful for caching data molecular memory device, storing operating system or BIOS files, and the like. Embedded system may include one or more input/output (I/O) interfaces that enable CPU to communicate with external devices and systems. I/O interface may be implemented by serial ports, parallel ports, radio frequency ports, optical ports, infrared ports and the like. Further, interface may be configured to communicate using any available protocol including packet-based protocols.

An illustrative example of a memory element in accordance with an embodiment of the present invention is akin to a widely used one transistor one capacitor (1T1C) memory element design, however, the present invention differs in that a molecular storage device is employed. In particular embodiments, the molecular storage device is implemented as a stacked structured formed subsequent to and above a semiconductor substrate having active devices formed therein. In other embodiments, the molecular storage device is implemented as a micron or nanometer sized hole in a semiconductor substrate have active devices formed therein. The molecular storage device is fabricated using processing techniques that are compatible with the semiconductor substrate and previously formed active devices in the semiconductor substrate. The molecular storage device comprises, for example, an electrochemical cell having two or more electrode surfaces separated by an electrolyte (e.g., a ceramic or solid electrolyte or by direct contact with a metal or semiconductor structure). Molecules of the invention having one or more oxidation states can be used for storing information and can be coupled to an electrode surface within an electrochemical cell. Illustrative examples of molecules that can be used for storing information including the compounds disclosed herein including, but not limited to, monomeric porphyrins, ferrocene-derivatived porphyrins, trimeric porphyrins, porphyrin polymers or triple-decker sandwich porphyrins, among other compounds.

When a word line is activated, an access transistor is placed in a conductive state thereby coupling molecular storage device to its associated bit line. In some cases, the signal generated by molecular storage is not sufficient to drive conventional logic devices. A sense amplifier detects the signal generated by molecular storage and amplifies that signal to an appropriate logic level (i.e., a signal compatible with other system logic). For example, using porphyrin molecules it is possible to construct storage devices with stable oxidation states at +0.55V, +0.48V, +0.39V, +0.17V, −0.05V and −0.18V. More or fewer stable oxidation states may be provided in a given embodiment. The storage molecules can be placed in a selected one of these oxidation states by application of an appropriate voltage to a bit line, and while activating an appropriate word line. Once the storage device is placed in the desired oxidation state, it will remain in that oxidation state for tens, hundreds, thousands or an indefinite number of seconds in particular applications.

During reading, a word line is activated and the storage device drives the bit line to a voltage that indicates its oxidation state. This is done in a manner very similar to that in which a capacitor performs the same operation. When the gate transistor is opened, the molecular storage device (MSD) is connected to the bit line. For example, the molecule has been written into an oxidized state, if the bit line is precharged to a value that is more negative than the oxidation potential of the molecule (with reference to the top metal of the MSD or counter electrode), and that molecule is in an oxidized state, current will flow from the molecule to the bit line (and electrons will flow from the bit line to the molecule) and the molecule is reduced once the transistor is closed. This will result in an accumulation of charge on the bit line, where the magnitude of the charge is determined by the number of molecules on the MSD and the oxidation state of each molecule (Q=nFN, Faraday's Law). The appearance of that charge will change the voltage on the bit line (V=Q/C), and that change in voltage can be distinguished by a voltage-sensing amplifier, as conventionally used in the art.

The bit line voltage during a read operation may vary from the oxidation state voltages due to parasitic effects and loading of the read circuitry, however, the circuitry is arranged to allow the stable oxidation states to be read distinctly. The bit line voltage may be read directly by external logic, or may be amplified to more conventional logic levels by sense amplifier. In particular implementations, the sense amplifier may include multiple reference points (e.g. a multi-state sense amplifier) so that it produces stable multi-valued signals on the bit line. Alternatively, sense amplifier may include analog-to-digital functionality that produces a plurality of logic-level binary outputs in response to the multi-value voltage signal read from a particular memory element.

In a particular example, the molecular storage device is configured as an electrochemical cell having a reference electrode and a working electrode coupled through an electrolyte. In one embodiment, the molecular storage device is configured as a stacked configuration for construction of a molecular storage device. In another embodiment, the molecular storage device is configured as a trench or “molehole” implementation. In the stacked implementation, the entire structure may be built on top of and electrically coupled to an electrode of an underlying semiconductor device. For example, conductive via or plug may reach down through passivation and planarization layers of a semiconductor device to make electrical contact with a source/drain region of an access transistor. Conductive plug may couple to a metal bond pad, or to the active region of a semiconductor device. In a particular example, plug comprises tungsten, but may be manufactured using any metal, alloy, silicide, or other material that is available for implementing electrical connectivity.

A working electrode of the invention comprises, for example, silicon, copper, aluminum, gold, silver, or other available conductor. While the working electrode may be a metal, it can also be a metaloxide, semiconductor or doped semiconductor. Other suitable materials include: Ti, Ta, TiO2, TaO₂, SiO₂, W, WOX and the like. The working electrode is preferably formed at the same time as other structures such as bond pads and interconnects for an integrated circuit. Processes and materials for forming plugs and electrodes are widely available in the semiconductor processing industry. In many integrated circuit processes, metal pads will be coated with insulating layer, which serves to protect and/or passivate working electrode. An insulating layer may be implemented as a deposited oxide, silicon nitride, or the like. An insulating layer is patterned to expose a portion of working electrode, preferably in the same operation used to expose portions of bonding pads of the integrated circuit. The exposed portion of working electrode defines an active area for the molecular storage device. It is contemplated that the present invention can be manufactured up through the formation and patterning of oxide using industry standard process flows.

A thin layer of storage molecules is formed on the active area of working electrode. This layer may range in thickness from 0.1 to 100 nanometers in illustrative examples. It is desirable to implement a layer as a self assembling monolayer (SAM). The active area of the molecules is lithographically defined by patterning layer over the conductive material. An extensive library of derivatized porphyrins (about 250 compounds) is available as storage molecules for attachment to metalelectrodes suitable for use in layer. These compounds may comprise many different architectures, for example, (1) monomeric porphyrins with different types of tethers, (2) ferrocene-derivatized porphyrins, (3) wing-shaped trimeric porphyrins, (4) porphyrin polymers, and (5) triple-decker sandwich porphyrins and polymers thereof. All of these porphyrinic architectures were found to form excellent quality self-assembled monolayers (SAMs). Once the molecules are attached, a thin (e.g., 50 to 200 nanometer) layer of metal, metaloxide or conductive electrolyte is applied to form electrolyte. A metal layer is deposited by evaporation, sputtering, or other deposition technique on to an electrolyte layer. A metal layer forms a reference electrode or counter electrode of the oxidation-reduction cell and comprises any well behaved electrochemical counter electrode material such as copper, silver, platinum and the like. Economics and semiconductor processes already developed will determine the metal of choice in a particular application.

In operation, storage molecules are attached to and electrically coupled to the working electrode. The electrolyte, which may be a liquid, gel, or solid that is chemically compatible with the storage molecules and other conductors and insulators used in the device. Electrolyte enables the transport of charge between the working and reference electrodes. For any given oxidation state and choice of storage molecules the electrochemical cell exhibits a distinctive electrochemical potential called the half-wave potential (E_{1/2}) or equilibrium potential. A given molecular storage device will have two, three, four, or more distinctive E_{1/2}'s depending on the particular storage molecules chosen. This offers the potential of manufacturing a single infrastructure including read/write logic, address decoders, interconnect circuitry and the like that can be customized at a late state of manufacture by selecting the particular storage molecules for the device. Some adjustment of the electronics will be required to compensate for the particular characteristics of the chosen storage molecule, however, the manufacturing advantages are clear.

One advantage of the stacked architecture is that the bottom surface of the capacitor forms the electrode surface, and storage molecules are able form monolayers on this surface. Also, electrolyte layer coats the storage molecules, essentially encapsulating them and protecting them from subsequent steps. Moreover, metal is never deposited directly onto the molecular layer, thereby preventing damage and other problems associated architectures that expose the storage molecules during or after processing to metal contamination. Further, the structure provides an easy way to implement a three-dimensional architecture in that subsequent layers of metal, insulator and the like are added after manufacture of the underlying semiconductor-based microelectronic devices while also preventing short-circuits.

In another embodiment, an electrochemical cell is formed in a trench structure, also called a “molehole” structure. A trench extends into substrate, through an overlying dielectric layer (e.g., oxide) and counter electrode. The walls of the trench are exposed and provide a surface contact to which storage molecules can be assembled. Storage molecules and electrolyte are added and the structure then be covered by a polymer to seal the array.

Each word line is connected to the counter electrode of the memory cells in a row, while each bit line is connected to the drains of the memory cells in a column. The ground voltage is connected to the source regions of the memory cells in a selected row. The row address decoder will activate all access transistors in a selected row thereby coupling each storage device in that row to their respective bit lines. The word lines and bit lines are manufactured using low temperature oxide deposition and metallization such that the integrity of the storage molecules is preserved. In may be preferred to use room temperature deposition of silicon dioxide and metals to form these structures. Existing molecules are able to withstand temperatures in excess of 400 degrees Celsius, allowing a broad range of temperatures for subsequent processes.

The trench architecture avoids the possibility of metal being deposited onto the molecular layer, thereby preventing damage and other problems associated with other proposed architectures. The inside of the trench forms the electrode surface, and molecules form SAMs on the inside of the cylinder, hence, the number of molecules can be increased by increasing the depth of the trench. The height of each layer of metal determines the height of the trench, thereby allowing easy adjustment of the effective area of the two terminals. Because the vertical dimension is used, many more molecules (30 times that of a simple crossbar for a 200 nm thick electrode) are available for writing/reading. This allows greatly enhanced sensitivity for a given cross-sectional area. In addition, the design easily accomplishes any variation in the relative sizes of each electrode. The effective capacitance of each junction is diminished by the removal of a large area of dielectric between the two metal plates at each intersection. This may have an effect on the overall wire capacitance; however, if the lines are sufficiently thick, the wire resistance will not be impacted significantly.

Another advantage of molecular storage devices is that molecular storage can readily be adapted to store multiple bits of data at each location by selection of the storage molecules. A cyclic voltammogram can be used to illustrate a current-voltage characteristic of a two-state monomeric porphyrin storage molecule. The peaks and valleys correspond to distinct oxidation states that can be used for storing information. Two peaks correspond to two distinct oxidization states and therefore a storage device that is capable of storing two states of information. Each oxidation state can be set or written to independently of the other. The lower portion of the cyclic curve corresponds to writing data to a molecular storage device whereas the upper portion of the cyclic curve corresponds to reading data from a molecular storage device.

In order to write a state into the storage device a voltage is applied to the bit line to create the desired oxidation state of the storage molecules. Typically, the voltage applied to the bit line will be somewhat more positive than the E_{1/2} of the molecule used in the MSD to compensate for resistive and capacitive losses in the writing circuitry. The losses associated with the writing circuitry are measurable and consistent, and so can be readily compensated. In a specific embodiment, the working electrode is held at a ground potential and the reference electrode is placed at a bias potential slightly below a peak in the IV curve. The bit lines are coupled to a write signal that indicates a first logic state with a voltage that when added to the bias potential is insufficient to cause oxidation of the storage molecules. The write signal indicates a second logic state with a voltage that when added to the bias potential is sufficient to cause oxidation of the storage molecules.

In a particular embodiment, the bias potential on reference electrode is set at 500 mV and the data signal applied to a bit line is either 0 or 300 mV. A 500 mV signal is insufficient to cause oxidation, whereas an 800 mv signal is sufficient to cause oxidation of the first discrete oxidation state. To write a second state to the same storage device, the bias potential on reference electrode is set above 800 mV. Once again, the data signal applied to a bit line is either 0 or 300 mV. An 800 mV signal is insufficient to cause oxidation, whereas an 1100 mV signal is sufficient to cause oxidation of the second discrete oxidation state. Hence, the first and second bits can be written subsequently.

After a write operation, the storage molecules will tend to remain at their oxidation state. In essence, the electrons added, or removed, by the write process are tightly bound to the storage molecules. In contrast, conventional solid state capacitors have electrons loosely captured in energy bands, where they are much more likely to escape. As a result, charge stored in storage molecules is much slower to leak away as compared to charge stored in a conventional capacitor. Once the storage molecules are written to a given oxidation state, they can be read by either voltage-mode sense amplifiers or current-mode sense amplifiers.

One characteristic of storage molecules is that if a voltage is applied to molecular storage device, the current magnitude will vary distinctively depending on the oxidation state of the storage molecules. If the storage molecules are already set at that particular oxidation state, no current will pass. For example, the storage device can be read by applying a voltage between the reference electrode and the working electrode more positive than the characteristic peak in the CV curve and comparing the current to a reference current source. A relatively large current indicates a first logic state while a relatively low current indicates a second logic state. In this manner, the oxidation state of the storage molecules after a write is determined by the state of the write signal coupled to the bit line. Although the exemplary implementations write each bit serially, it is contemplated that the molecules can be written to and/or read from in parallel.

This reading methodology is partially or wholly destructive in that application of a potential during reading will alter the oxidation state of at least some storage molecules the storage device. Accordingly, a read operation is desirably followed by a write back to the molecular storage device to restore the oxidation state of the storage molecules.

Molecular storage promises great expansion in information density because storage molecules can be designed to with almost any number of distinct oxidation states. As each oxidation state is capable of storing one bit if information the information density of a memory array increases dramatically. By implementing read/write logic, sense amplification mechanisms and the like that support molecular storage the present invention enables the use of these properties in practical memory devices.

At any particular voltage the current is determined by the various oxidation states. Accordingly, reading a storage device can be accomplished by applying a particular voltage to the storage device, measuring the current, and mapping the measured current to an appropriate logic state for the number of bits stored in the storage device.

The invention is also applicable for parallel current sensing. The cell current (ICELL) is coupled to a current-mode sense amplifier. Cell current may be directly from a bit line, or may instead by pre-amplified. A pre-amplifier may be included within each current mode sense amplifier or provided separately. Each sense amplifier has a unique current referenced, which indicates a threshold current value for a particular logic state. Each sense amplifier generates a binary signal indicating whether the cell current exceeded the reference current. Logic encoder receives the binary signals and maps them to logic signals, which may include level shifting, inverting, or processing with other combinatorial logic to meet the needs of a particular application.

Multi-electrode molehole arrays are well suited for use as memory elements in molecular based electronic devices and may be utilized in implementing the molecular memory arrays and devices of the present invention. In “molecular memory” elements redox-active molecules (molecules having one or more non-zero redox states) coupled to an electrode (e.g. the working electrode) in a molehole are used to store bits (e.g. in certain embodiments, each redox state can represent a bit or a combination of bits). The redox-active molecule attached to the electrode (e.g., silicon or germanium) forms a storage I cell capable of storing one or more bits in various oxidation states. In certain embodiments, the storage cell is characterized by a fixed working electrode electrically coupled to a “storage medium” comprising one or more redox-active molecules and having a multiplicity of different and distinguishable oxidation states. Data is stored in the (preferably non-neutral) oxidation states by the addition or withdrawal of one or more electrons from said storage medium via the electrically coupled electrode. The oxidation state of the redoxactive molecule(s) can be set and/or read using electrochemical methods (e.g. cyclic voltammetry), e.g., as described in U.S. Pat. Nos. 6,272,038, 6,212,093, and 6,208,553 and PCT Publication WO 01/03126, the disclosures of which are herein incorporated by this reference in their entirety. A molehole array comprising a plurality of moleholes (electrochemical cells) can provide a high capacity, high density memory device.

Because group N elements, in particular silicon and germanium, are commonly used in electronic chip fabrication, they readily lend themselves to the fabrication of molecular memory chips compatible with existing processing/fabrication technologies. In addition, details on the construction and use of storage cells comprising redox-active molecules can be found, in U.S. Pat. Nos. 6,272,038, 6,212,093, and 6,208,553 and PCT Publication WO 01/03126.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

A number of references have been cited, the entire disclosures of which are incorporated herein by reference.

What is claimed is:

1. A self-contained charge storage molecule comprising:
(a) one or more reducible functional species (ReS);
(b) one or more oxidizable functional species (Ox); and
(c) one or more ion conducting moiety (IC).

2. The self-contained charge storage molecule of claim 1, wherein each of the one or more reducible species components (ReS), the one or more oxidizable species components (Ox), and the one or more ion conducting moiety components (IC) are chemically attached.

3. The self-contained charge storage molecule of claim 2, wherein said attachment is a covalent bond.

4. The self-contained charge storage molecule of claim 2, wherein said attachment is ionic.

5. The self-contained charge storage molecule of claim 1, wherein the reducible functional species is of the formula: 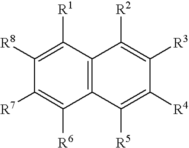
wherein each of R¹-R⁸ is independently —H; —OH, —CN, halogen, —N(C₁-C₁₀)alkyl(C₁-C₁₀)alkyl, —(C₁-C₁₀)alkyl (including C₁-C₁₀ perfluoroalkyl), —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; or adjacent R groups can form a 3-8 membered ring optionally substituted.

6. The self-contained charge storage molecule of claim 5, wherein the reducible functional species is of the formula: 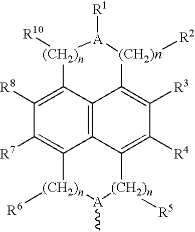
wherein
A is independently N, O, S, Se, Te, CH, or CH₂;
R¹-R¹⁰ are each independently —H; —OH, —CN, halogen, —N(C₁-C₁₀)alkyl(C₁-C₁₀)alkyl, —(C₁-C₁₀)alkyl (including C₁-C₁₀ perfluoroalkyl); —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; optionally R², R⁵, R⁶, and R¹⁰ can be a ═O group;
n is 0-5; and [symbol]
is an attachment, preferably covalent to the oxidizable species or the ionic conducting electrolyte.

7. The self-contained charge storage molecule of claim 6, wherein at least one A is —N—.

8. The self-contained charge storage molecule of claim 6, wherein both A groups are —N—.

9. The self-contained charge storage molecule of claim 6, wherein at least one of R², R⁵, R⁶ and R¹⁰ is a ═O group.

10. The self-contained charge storage molecule of claim 6, wherein each of R², R⁵, R⁶, and R¹⁰ is a ═O group.

11. The self-contained charge storage molecule of claim 6, wherein R¹ is an aryl group.

12. The self-contained charge storage molecule of claim 6, wherein at least one of R³, R⁴, R⁷, and R⁸ is selected from the group consisting of —CN, halogen, or perfluoroalkyl.

13. The self-contained charge storage molecule of claim 6, wherein each of R³, R⁴, R⁷, and R⁸ is —H.

14. The self-contained charge storage molecule of claim 5, wherein the reducible functional species is of the formula:

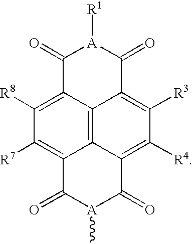

15. The self-contained charge storage molecule of claim 14, wherein at least one A group is —N—.

16. The self-contained charge storage molecule of claim 14, wherein both A groups are —N—.

17. The self-contained charge storage molecule of claim 14, wherein at least one of R³, R⁴, R⁷, and R⁸ is —H.

18. The self-contained charge storage molecule of claim 14, wherein at least one of R³, R⁴, R⁷, and R⁸ is selected from the group consisting of —CN, halogen, or perfluoroalkyl.

19. The self-contained charge storage molecule of claim 14, wherein each of R³, R⁴, R⁷, and R⁸ is —H.

20. The self-contained charge storage molecule of claim 14, wherein the reducible species is of the formula: 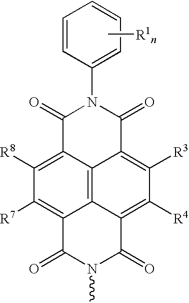
wherein each R¹ is independently —H; —OH, —CN, halogen, —N(C₁-C₁₀)alkyl(C₁-C₁₀)alkyl, —(C₁-C₁₀)alkyl (including perfluoroalkyl); —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)_{X}-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; optionally R², R⁵, R⁶, and R¹⁰ can be a ═O group; and
n is 0-5.

21. The self-contained charge storage molecule of claim 1, wherein the reducible species is of the formula: 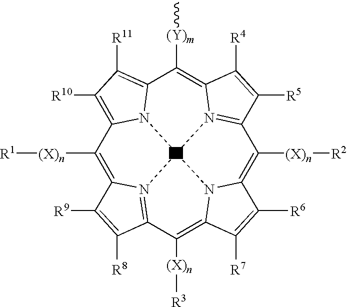
wherein:
M is Zn, Mg, Cd, Hg, Cu, Ag, Au, Ni, Pd, Pt, Co, Rh, Ir, Mn, B, Al, Ga, Pb, and Sn;
X and Y are each independently a bond or NH, O, S, Se, Te, or CH₂;
R¹, R², and R³ are each independently aryl, benzyl, cycloalkyl, alkyl, halogen, alkoxy, alkylthio, perfluoroalkyl, perfluoroaryl, pyridyl, cyano, thiocyanato, nitro, amino, alkylamino, acyl, sulfoxyl, sulfonyl, imido, amido, and carbamoyl;
each R⁴, R⁵, R⁶, R⁷, R⁸, R⁹ and R¹⁰ is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), and —SO₂NH₂;
each n is independently 0-3;
m is 0-5; [symbol]
is an attachment to the reducible species or the ionic conducting electrolyte.

22. The self-contained charge storage molecule of claim 1, wherein the oxidizable species is of formula: 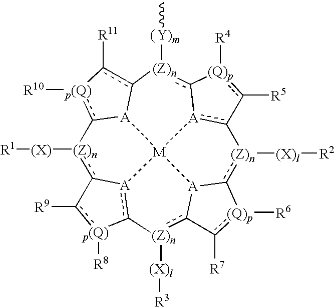
wherein:
each A is independently N, O, S, Se, Te, CH, or CH₂;
M is absent or is a metal including, but not limited to, Zn, Mg, Cd, Hg, Cu, Ag, Au, Ni, Pd, Pt, Co, Rh, Ir, Mn, B, Al, Ga, Pb, and Sn;
each Z is independently N, O, S, Se, Te, C;
X and Y are each independently a bond or NH, O, S, Se, Te, or CH₂;
R¹, R², and R³ are each independently aryl, benzyl, cycloalkyl, alkyl, halogen, alkoxy, alkylthio, perfluoroalkyl, perfluoroaryl, pyridyl, cyano, thiocyanato, nitro, amino, alkylamino, acyl, sulfoxyl, sulfonyl, imido, amido, and carbamoyl;
each R⁴, R⁵, R⁶, R⁷, R⁸, R⁹ and R¹⁰ is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂;
each Q is independently a C or a heteroatom;
each n is independently 0-3;
m is 0-5;
each l is independently 0-5;
each p is independently 0-3; and [symbol]
is an attachment to the reducible species or the ionic conducting electrolyte.

23. The self-contained charge storage molecule of claim 22, wherein at least one A is —N—.

24. The self-contained charge storage molecule of claim 22, wherein each A group is —N—.

25. The self-contained charge storage molecule of claim 22, wherein M is Zn.

26. The self-contained charge storage molecule of claim 22, wherein at least one Z group is —C—.

27. The self-contained charge storage molecule of claim 22, wherein each Z group is —C—.

28. The self-contained charge storage molecule of claim 22, wherein each of X and Y is a bond.

29. The self-contained charge storage molecule of claim 22, wherein at least one of R¹, R², and R³ is an optionally substituted aryl group.

30. The self-contained charge storage molecule of claim 29, wherein each of R¹, R² and R³ is an optionally substituted aryl group.

31. The self-contained charge storage molecule of claim 29, wherein the aryl group is substituted with at least one ferrocene group.

32. The self-contained charge storage molecule of claim 22, wherein at least one of R⁴, R⁵, R⁶, R⁷, R⁸, R⁹ and R¹⁰ is —H.

33. The self-contained charge storage molecule of claim 22, wherein each of R⁴, R⁵, R⁶, R⁷, R⁸, R⁹ and R¹⁰ is —H.

34. The self-contained charge storage molecule of claim 22, wherein Q is C.

35. The self-contained charge storage molecule of claim 22, wherein n is 1.

36. The self-contained charge storage molecule of claim 22, wherein m is 0.

37. The self-contained charge storage molecule of claim 22, wherein 1 is 0.

38. The self-contained charge storage molecule of claim 22, wherein p is 1.

39. The self-contained charge storage molecule of claim 22, wherein the oxidizable species is of formula:

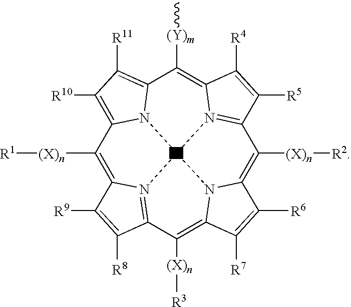

40. The self-contained charge storage molecule of claim 22, wherein the oxidizable species is of formula:

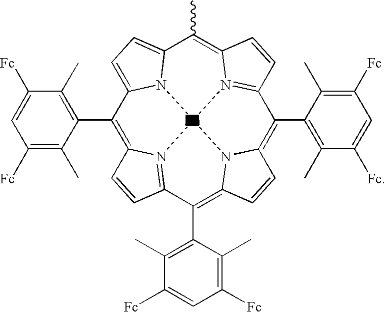

41. The self-contained charge storage molecule of claim 1, wherein the ionic conducting electrolyte is either a covalently bound cationic or anionic species with requisite counter ions for charge neutrality.

42. The self-contained charge storage molecule of claim 41, wherein the ionic conducting electrolyte has the formula: 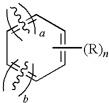
wherein each R is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —(P((C₁-C₁₀)alkyl)₃)+, —SO₃—, —SO₂NH₂;
n is 0-4;
( [symbol] )ₐ is an attachment to either the oxidizable species or the reducible species; and
( [symbol] )_{b} is an optional attachment to either the oxidizable species or the reducible species.

43. The self-contained charge storage molecule of claim 41, wherein n is 4 and two R groups are aryl substituted aryl groups and two R groups are SO₃— with an appropriate counter ion.

44. The self-contained charge storage molecule of claim 41, wherein n is 4 and two R groups are aryl substituted aryl groups and two R groups are —(P((C₁-C₁₀)alkyl)₃)⁺ with an appropriate counter ion.

45. The self-contained charge storage molecule of claim 42, wherein the ionic conducting electrolyte has the formula: 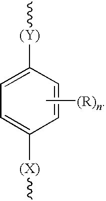
wherein X and Y are linkages between the oxidizable species and the reducible species, wherein the linkages vary in type, length and functionality.

46. The self-contained charge storage molecule of claim 42, wherein the ionic conducting electrolyte has the formula:

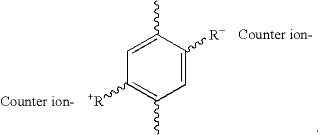

47. The self-contained charge storage molecule of claim 46, wherein at least one R group is a —(P((C₁-C₁₀)alkyl)₃)⁺ with an appropriate counter ion.

48. The self-contained charge storage molecule of claim 46, wherein each R group is a —(P((C₁-C₁₀)alkyl)₃)⁺ with an appropriate counter ion.

49. The self-contained charge storage molecule of claim 42, wherein the ionic conducting electrolyte has the formula: 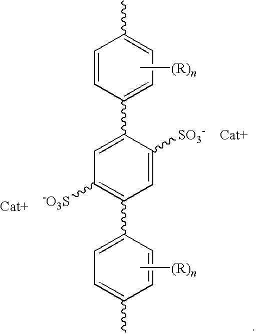
wherein Cat⁺ is any suitable cation and ( [symbol] ) is an optional attachment to either the oxidizable species or the reducible species, which vary in type, length and functionality.

50. The self-contained charge storage molecule of claim 42, wherein the ionic conducting electrolyte has the formula:

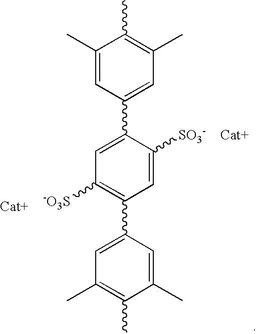

51. The self-contained charge storage molecule of claim 42, wherein the ionic conducting electrolyte has the formula: 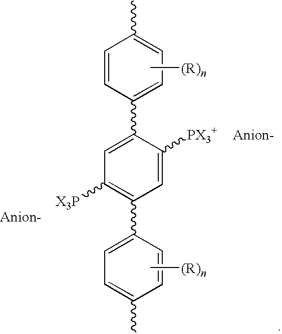
wherein each ( [symbol] ) is an optional covalent linkage or a direct bond, which vary in type, length and functionality.

52. A self-contained charge storage molecule of formula:

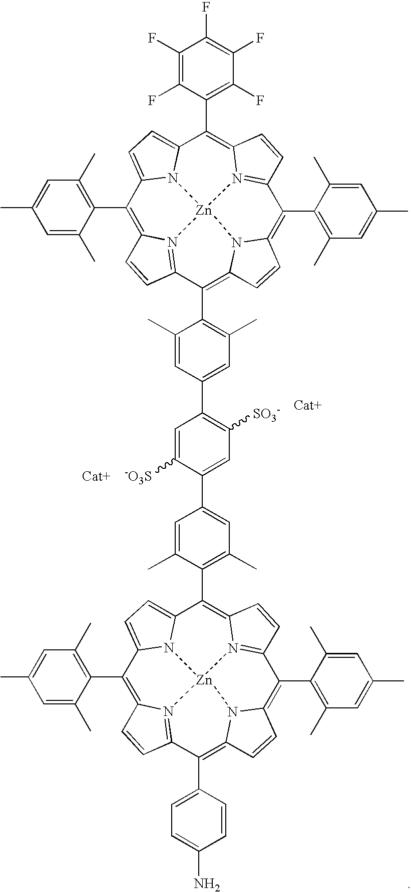

53. A self-contained charge storage molecule of formula:

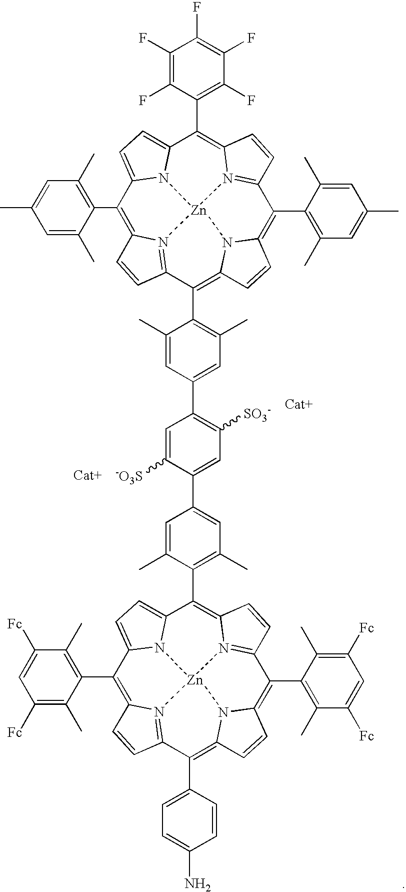

54. A self-contained charge storage molecule of formula:

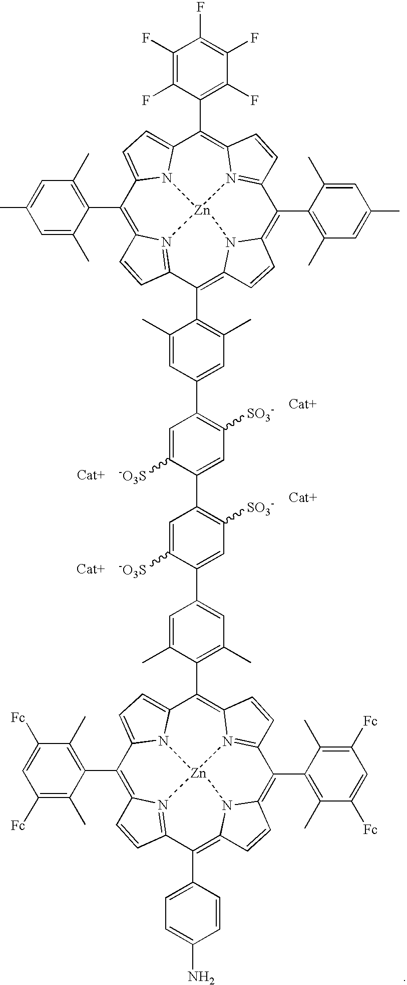

55. A self-contained charge storage molecule of formula:

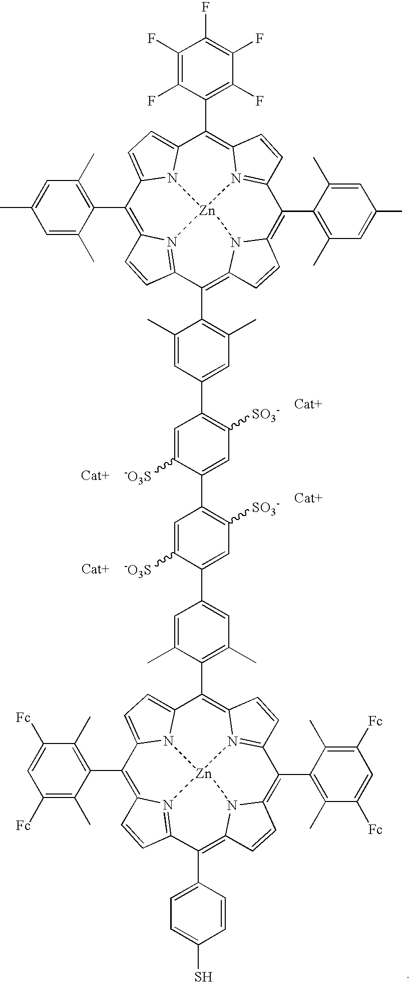

56. A self-contained charge storage molecule of formula:

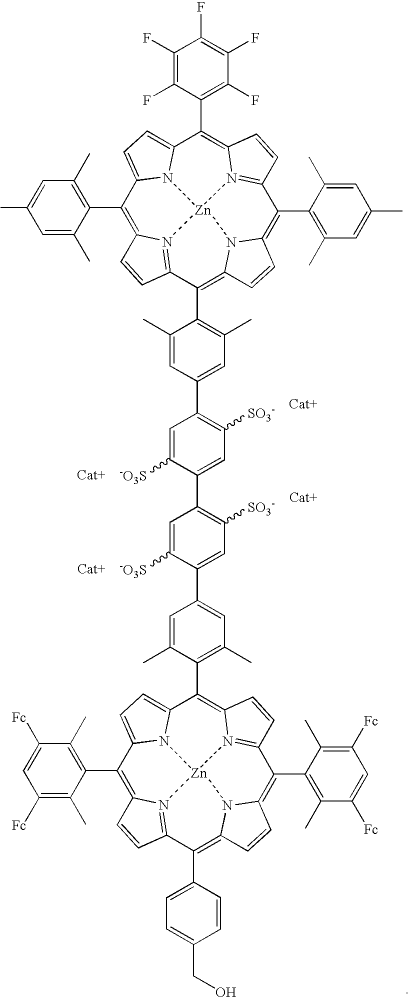

57. A self-contained charge storage molecule of formula:

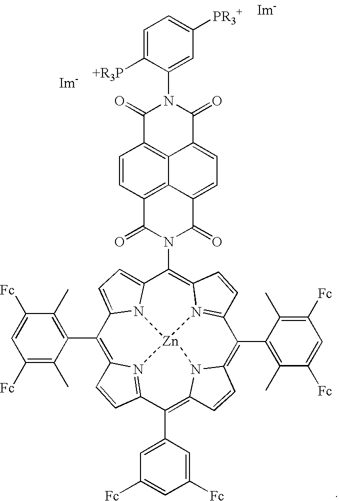

58. A self-contained charge storage molecule of formula:

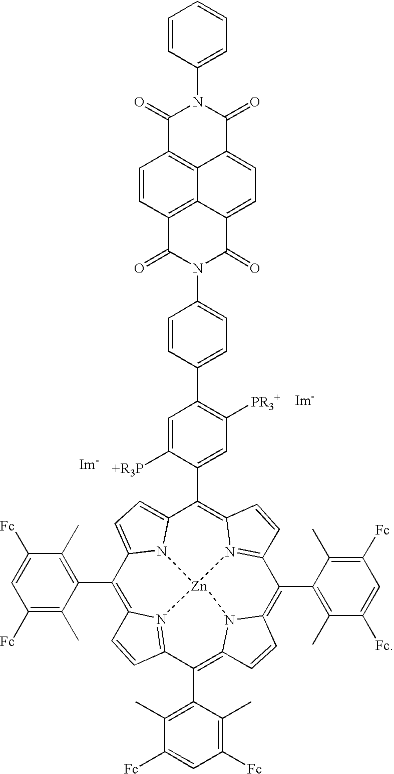

59. A molecular capacitor characterized in that: conductive plates are separated by one or more molecules comprised of an oxidizable component, a reducible component, and an ionic conducting electrode.

60. A molecular charge storage device, comprising:
a working electrode and a counter electrode configured to afford electrical capacitance; and
one or more self-contained charge storage molecules positioned between said working and counter electrodes, said self-contained charge storage molecules comprised of one or more reducible functional species (ReS); one or more oxidizable functional species (Ox); and one or more ion conducting moiety (IC).

61. The molecular charge storage device of claim 60 wherein the one or more reducible functional species are chemically bound to the counter electrode and the counter electrode is negatively-biased.

62. The molecular charge storage device of claim 60 wherein the one or more oxidizable functional species are chemically bound to the working electrode, and the working electrode is positively-biased.

63. The molecular charge storage device of claim 60 wherein the one or more ion conducting moiety exhibits no appreciable electrical conductivity, and is in sufficient proximity to the oxidizable and reducible functional species such that both the oxidizable and reducible species develop charge.

64. The molecular charge storage device of claim 60, wherein the reducible functional species is of the formula: 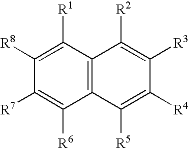
wherein each of R¹-R⁸ is independently —H; —OH, —CN, halogen, —N(C₁-C₁₀)alkyl(C₁-C₁₀)alkyl, —(C₁-C₁₀)alkyl (including perfluoroalkyl); —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; or adjacent R groups can form a 3-8 membered ring optionally substituted.

65. The molecular charge storage device of claim 60, wherein the reducible functional species is of the formula: 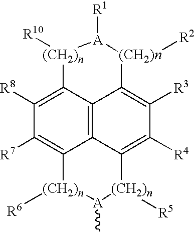
wherein
A is independently N, O, S, Si, Se, Te, CH, or CH₂;
R¹-R¹⁰ are each independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; optionally R², R⁵, R⁶, and R¹⁰ can be a ═O group;
n is 0-5; and [symbol]
is an attachment, preferably covalent to the oxidizable species or the ionic conducting electrolyte.

66. The molecular charge storage device of claim 60, wherein the reducible functional species is of the formula:

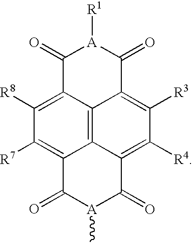

67. The molecular charge storage device of claim 60, wherein the reducible species is of the formula: 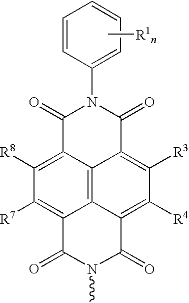
wherein each R¹ is independently —H; —OH, —CN, halogen, —N(C₁-C₁₀)alkyl(C₁-C₁₀)alkyl, —(C₁-C₁₀)alkyl (including perfluoroalkyl); —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), (with all alkyl (including heteroalkyl and cycloalkyl and cycloheteroalkyl) and aryl (including heteroaryl) including optionally and independently selected substitutents), —SO₂NH₂; optionally R², R⁵, R⁶, and R¹⁰ can be a ═O group; and
n is 0-5.

68. The molecular charge storage device of claim 60, wherein the reducible species is of the formula: 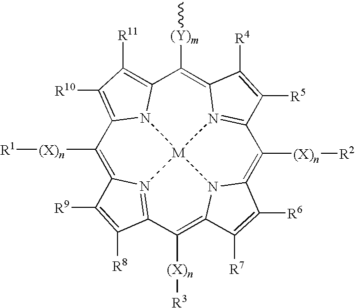
wherein:
M is Zn, Mg, Cd, Hg, Cu, Ag, Au, Ni, Pd, Pt, Co, Rh, Ir, Mn, B, Al, Ga, Pb, and Sn;
X and Y are each independently a bond or NH, O, S. Se, Te, or CH₂;
R¹, R², and R³ are each independently aryl, benzyl, cycloalkyl, alkyl, halogen, alkoxy, alkylthio, perfluoroalkyl, perfluoroaryl, pyridyl, cyano, thiocyanato, nitro, amino, alkylamino, acyl, sulfoxyl, sulfonyl, imido, amido, and carbamoyl;
each R⁴, R⁵, R⁶, R⁷, R⁸, R⁹ and R¹⁰ is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂;
each n is independently 0-3;
m is 0-5; [symbol]
is an attachment to the reducible species or the ionic conducting electrolyte.

69. The molecular charge storage device of claim 60, wherein the oxidizable species is of formula: 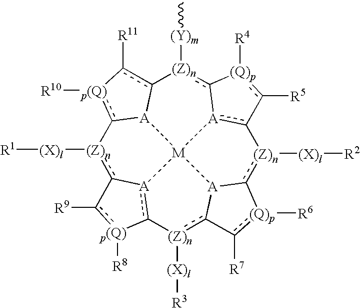
wherein:
each A is independently N, O, S, Se, Te, CH, or CH₂;
M is absent or is a metal including, but not limited to, Zn, Mg, Cd, Hg, Cu, Ag, Au, Ni, Pd, Pt, Co, Rh, Ir, Mn, B, Al, Ga, Pb, and Sn;
each Z is independently N, O, S, Se, Te, C;
X and Y are each independently a bond or NH, O, S, Se, Te, or CH₂;
R¹, R², and R³ are each independently aryl, benzyl, cycloalkyl, alkyl, halogen, alkoxy, alkylthio, perfluoroalkyl, perfluoroaryl, pyridyl, cyano, thiocyanato, nitro, amino, alkylamino, acyl, sulfoxyl, sulfonyl, imido, amido, and carbamoyl;
each R⁴, R⁵, R⁶, R⁷, R⁸, R⁹ and R¹⁰ is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —SO₂NH₂;
each Q is independently a C or a heteroatom;
each n is independently 0-3;
m is 0-5;
each l is independently 0-5;
each p is independently 0-3; and [symbol]
is an attachment to the reducible species or the ionic conducting electrolyte.

70. The molecular charge storage device of claim 60, wherein the oxidizable species is of formula:

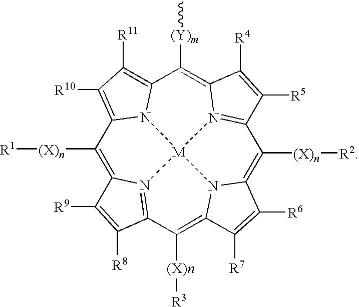

71. The molecular charge storage device of claim 60, wherein the oxidizable species is of formula:

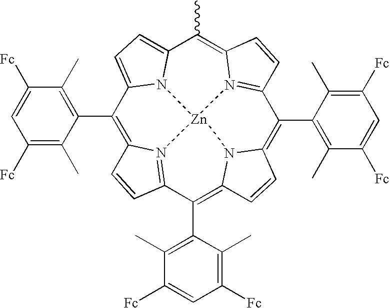

72. The molecular charge storage device of claim 60, wherein the ionic conducting electrolyte has the formula: 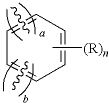
wherein each R is independently —H; —OH, —N(C₁-C₁₀)alkyl(C₁-C₁₀)alkyl, —(C₁-C₁₀)alkyl; —O(C₁-C₁₀)alkyl, —O—C(O)(C₁-C₁₀)alkyl, —NH(CH₂)ₘ(C₁-C₁₀)alkyl, —O—CF₃, —O-benzyl, —O—(CH₂)ₘCH((C₁-C₁₀)alkyl(C₁-C₁₀)alkyl), —(C₂-C₁₀)alkenyl, —(C₂-C₁₀)alkynyl, —(C₃-C₁₀)cycloalkyl, —(C₈-C₁₄)bicycloalkyl, —(C₅-C₁₀)cycloalkenyl, —O—(C₂-C₁₀)alkenyl, —O—(C₂-C₁₀)alkynyl, —O—(C₃-C₁₀)cycloalkyl, —O—(C₈-C₁₄)bicycloalkyl, —O—(C₅-C₁₀)cycloalkenyl, —O-heteroaryl, -heteroaryl, -aryl, —(C₃-C₁₀)heterocyclealkyl, —O—(CH₂)ₙ-aryl, —O—(C₃-C₁₀)heterocycloalkyl, —NHC(O)(C₁-C₁₀)alkyl, —NHC(O)NH(C₁-C₁₀)alkyl, —NH(aryl), —N═C(aryl), —(P((C₁-C₁₀)alkyl)₃)+, —SO₃—, —SO₂NH₂;
n is 0-4;
( [symbol] )ₐ is an attachment to either the oxidizable species or the reducible species; and
( [symbol] )_{b} is an optional attachment to either the oxidizable species or the reducible species.

73. The molecular charge storage device of claim 60, wherein the ionic conducting electrolyte has the formula: 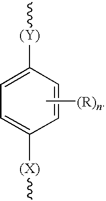
wherein X and Y are linkages between the oxidizable species and the reducible species, wherein the linkages vary in type, length and functionality.

74. The molecular charge storage device of claim 60, wherein the ionic conducting electrolyte has the formula: 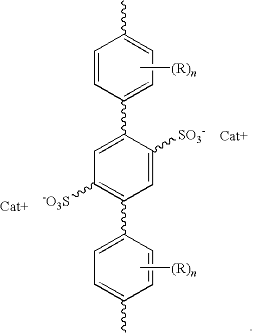
wherein Cat⁺ is any suitable cation and ( [symbol] ) is an optional attachment to either the oxidizable species or the reducible species, which vary in type, length and functionality.

75. The molecular charge storage device of claim 60, wherein the ionic conducting electrolyte has the formula:

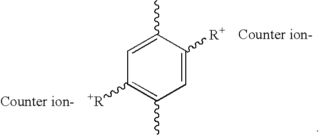

76. A memory element, comprising:
a switching device,
a bit line and a word line coupled to the switching device; and
a molecular storage device coupled to the switching device, the molecular storage device comprised of:
molecules positioned between a first electrode and a second electrode, and said molecules are comprised of one or more reducible functional species (ReS); one or more oxidizable functional species (Ox); and one or more ion conducting moiety (IC).

77. The memory element of claim 76 further comprising:
an attachment layer formed on the first electrode, and
an electrolyte layer formed on the attachment layer,
wherein the attachment layer has an opening containing the molecules, and the molecules are electronically coupled to the second electrode and the electrolyte layer.

78. A memory array, comprising:
an array of memory elements, wherein at least some of the memory elements comprise:
a switching device,
a bit line and a word line coupled to the switching device; and
a molecular storage device coupled to the switching device, the molecular storage device comprised of: molecules positioned between a first electrode and a second electrode, and said molecules are comprised of one or more reducible functional species (ReS); one or more oxidizable functional species (Ox); and one or more ion conducting moiety (IC).

79. An electronic device comprising:
a molecular memory element comprised of: a switching device, a bit line and a word line coupled to the switching device; and a molecular storage device coupled to the switching device, the molecular storage device comprised of:
molecules positioned between a first electrode and a second electrode, and said molecules are comprised of one or more reducible functional species (ReS); one or more oxidizable functional species (Ox); and one or more ion conducting moiety (IC); and
one or more functional components coupled to said molecular memory element.

80. The electronic device of claim 79 wherein said one or more function components may be comprised of solid state electronic devices, molecular electronic devices and combinations thereof:
molecules positioned between a first electrode and a second electrode, and said molecules are comprised of one or more reducible functional species (ReS); one or more oxidizable functional species (Ox); and one or more ion conducting moiety (IC).
